SEQUENCE LISTING (72 sequences)

SEQ ID NO: 1 - DNA, 610 bp, human polyomavirus
aaatgttcytccrgttcttcatataacaaacactgccacaacwgtgytgctygatgaatttggtgttggdccactttgcaaaggkgmcaayttrtayttgtcagctgtkgatgtbtgtggcatgtttachaacagrtcwgrytcccarcartggagaggrctbtcyagatattttaaggtkcarcthagraaaagragrgttaaaaacccctacccaatttctttycttcttacwgayytaatwaacagraggacycctagagttgatggkcagcctatgtatggcatggatgctcargtdgargaggttagrgtttttgagggracagaggrrcttccaggggacccrgayatgatgagataygttgacaratatggacagttgcaracmaaratgctgtratbamaagcctttattgtraywtgcagwrmawtttaataaagtrtaaccagytkhacttrrywkttgharttatttbgggggaggkgtytttggttttttraarcattgraarcctttacaratgtgawadgtrcagtkttcctgygtgyctgcaccagaggcttctgagacctgggaadagyattgtgaytgdgattcagtgcttga

SEQ ID NO: 2 - DNA, 610 bp, human polyomavirus
aaatgtycctccagtkctkcatatmrcaaacactgccacaacagtgctgcttgatgaatttggkgttggrccactttgcaaaggtgacaacttgtatttgtcagctgttgatgtttgtggcatgtttactaacagatctggytcccagcagtggagaggwctstccagatattttaaggtkcagytaagaaaaagragggtyaaaaacccctacccaatytctttccttcttactgayttaattaacagaaggacccctagagttgatggkcarccwatgtatggcatggatgctcaratagaggaggttagagtwtttgagggvacagagcaacttccaggggacccagatatgatgagatatgttgacaratatggacagttgcaracaaaratgctrtaatyaamagcctttattgtaawatgcartacatttkaataaagtrtraccagctttactttacayttgsagttattttgggggaggggtttttggtttyttraaacatkgaaarcctttacaratgtgatargtgcartgytcctgwgtgtctgcaccagaggcttctgagacctgggaaragcattgtgattgagattcagtgcttga

SEQ ID NO: 3 - DNA, 610 bp, human polyomavirus
aaatgttcctccagtycttcayataacraacactgchacaacagtgctgcttrrtgaatytggtgttgggccactttgcaaaggtgacaacttgtatyygtcagctgtkgatgtttgtggmatgytyactaacagrtctggttcccarcagtggagaggrctgtycagatattttaargttcarytragaaaargrrgggttaaaaacccctacccaatttctttycttcttactgatytrattaacagaagracccctagagtkgatgggcagcctatgtatggcatggrtgmycaggtagaggaggttagagtwtttkwgggracagaggaacttccwggggrcccagayatratgagayatgttgacaratatggacarttgcaracaaaratgctgtaayvaaaavcctttattgtaayaygcaktacattktaataaavyvtaaccagyttkactttdcagttgcagttayyttggggrrggkgtytttsgkyttttgaarcaytgraarcctttacadayrtgawwrgtgcagtsttcctgtgtgtctgcaccagaggyttctgagacctgggaakarcattgygattgagatycagtgcttga

SEQ ID NO: 4 - DNA, 610 bp, human polyomavirus
aaatgttcctccagttcttcatataacaaacwctgccacaacagtkctgcttgatgaatttggtgttgggcctctttgcaaaggtgacaacttgtatttgtcagctgttgatgtttgtggaatgtttactaacagatctggttcccagcagtggagaggrytgtccagatattttaaggttcagctaagaaaaaggagggttaaaaacccytacccaatttctttccttcttactgatttgattaacagaaggacccctagagttgatgggcagcctatgtatggtatggatgctcaggtagaggaggttagagtttttgaggggacagaggaacttccaggggacccagatatgatgagatatgttgacaratatggacagttgcaaacaaagatgctgtaakcaaaagcctttattgtaatvtgcagtacattttaataaagtataacsagctttactttacagttgcagtcattttgggggaggggtytttggttttttgaaacaytgaaagcctttacaratgtgatargtgcagtgttcctgtgtgtctgcaccagaggcttctgagacctgggaagagcattgtgattgagattcagtgcttga

SEQ ID NO: 5 - DNA, 610 bp, human polyomavirus
aaatgttcctccagttcttcatataacaaacactgccacaacagtgctgcttgatgaatttggtgttgggcctctttgcaaaggtgacaacttgtatttgtcagctgttgatgtttgtggratgtttactaacagatctggttcccagcagtggagaggactgtccagatattttaaggttcagctgagaaaaaggagggttaaaaacccctacccaatttctttccttcttactgatttgattaacagaaggacccctagagttgatggrcagcctatgtatggtatggaygctcaggtagaggaggttagagtttttgaggggacagaggaacttccaggggacccagacatgatgagatatgttgacagatatggacagttgcaaacaaagatgctgtaatcaaaagcctttattgtmatatgcagtacattttaataaagtataaccagctttactttacagttgcagtcattttgggggaggggtttttggttttttgaaacattgaaagcctttacagatgtgataagtgcagtgttcctgtgtgyctgcaccagaggcttctgagacctgggaaaagcattgtgattgagattcagtgcttga

SEQ ID NO: 6 - DNA, 610 bp, human polyomavirus
aaatgttcctccagttcttcatataacaaacactgccacaacagtgctgcttgatgaatttggygttgggccactttgcaaaggtgacaacttgtayttgtcagctgttgatgtttgtggcatgtttactaacagatctggttcccarcagtggagaggactgtcyagatattttaaggttcagctaagaaaaaggagggttaaaaacccctacccaatttcttttcttcttactgatttaatyaacagaaggacccctagagttgatgggcagcctatgtatggcatggatgctcaggtagaggaggttagagtttttgagggracagaggaacttccaggggacccagacatgatgagatatgttgacagatatggacagttgcaracaaaratgctgtaatsaaaagcctttattgtaatatgcagtacattttaataaagtataaccagcttyacttgacagttgcagttattttgggggaggggtttttggttttttgaaacattgaaagcctttacagatgtgatargtgcagtgttcctgtgtgtctgcaccagaggcttctgagacctgggaagagcattgtgattgagattcagtgcttga

SEQ ID NO: 7 - DNA, 610 bp, human polyomavirus
aaatgttcctccagttcttcatataacaaacacygchacaacvgtgcygcttgatgaatttggtgttggdccactttgcaaaggtgacaayttgtatytgtcagctgttgatgtttgtggcatgtttactaayagatctggttcccagcagtggagdrgrytgtccagatattttaaggttcagytaagraaaagrarggttaaraayccstacccaatttcyttccttcttactgayttratwaacagaagracccctagarttgatgggcagcctatgtatggcatggatgctcaratagaggaggtyagagtttttgaggggacagaggaactyccaggggacccwgacatgatragatatgttgacarrtatggrcarttgcaracaaaratgctgtaatyaaaagcctttattgtaayatgcagtacatwtbaataaartmwaaccagcttwactttahasttgcwgtyaytttgggggrggggtytttggttttttgaaacattgaaagcctttacaratgtgatargtgcagtgttcctgtgtgtctgcacyagaggcttctgagacytgggaaragcattgtgattgagattcagtgcttga

SEQ ID NO: 8 - DNA, 610 bp, human polyomavirus
aaatgttcctccagttcttcatatwacaaayactgccacaacagtgctgcttkatgaatttggygttgggccactttgcaaaggtgacaacttgtatttgtcagctgtwgatgtttgtggratgtttachaacagrtstggttcccagcagtggagaggactgtcyagatayttyaaggtkcagctsagaaaaagragggttaaaaacccctacccaatttctttcctycttactgatttrattamhagraggacccctagrgttgayggrcagcctatgtayggtatggatgctcaggtagaggaggttagagtttttgaggggacagaggaacttcyaggggacccagacatgatgagataygttgacagatatggacagtygcaracaaagatgctgtrrysaaaagcctttattgtaatatgcagtacatttyaataaagtryaaccagsttyactttwcagttgcagtyattttgggggaggggtttttggttttttgaaacatygraarcctttacagatgtgawaagtgcastgttcctgtgtgtytgcaccagvggcttctgagacctgggarragyattgtgaytgrgattcagtgcttga

SEQ ID NO: 9 - DNA, 610 bp, human polyomavirus
aaatgtycctccagttcttcatataacaaacactgccacaacagtgctgcttgatgaatttggtgttgggccactttgcaaaggtgacaacttgtatttgtcagctgttgatgtttgtggratgtttactaacagrtctggttcccagcagtggagaggactgtccagatattttaaggttcagytgagaaaaaggagggtyaagaacccctacccaatttctttccttcttactgatttgattaacagaaggacccctagagttgatgggcagcctatgtatggtatggatgctcaggtagaggaggttagagtttttgaggggacagaggaacttccaggggacccagacatgatgagatatgttgacagatatggacagttgcaaacaaagatgctgtaatcaaaagcctttattgtaatatgcagtacattttaataaagtatarccagctttactttacagttgcagtcattttgggggaggggtttttggttttttaaaacattgaaagccttkacagatgtgataagtgcagtgttcctgtgtgtctgcaccagaggcttctgagacctgggaaaagcattgtgattgagattcagtgcttga

SEQ ID NO: 10 - DNA, 610 bp, human polyomavirus
aaatgttcctccagtyctycatataacraacactgccacaacagtgctgcttgatgaatttggtgttggvcctctttgcaaaggtgacaacttgtatttgtcagctgttgatgtttgtggaatgtttactaacagatctggttcccagcagtggagaggactgtccagatattttaaggttcagctgagaaaaaggagggttaaaaacccctacccaatttctttcctkcttacwgatttrattaacagaaggacccctagagttgatggrcagcctatgtatggtatggatgctcaggtagaggaggttagagtttttgagggracagaggaacttccaggggacccagacatgatgaratatgttgacagatatggacagttgcaaacaaagatgctgtaatmaaaagcctttattgtaatatgcagtacattttaataaagtwtaaccagctttactttasakttkcagtyattttgggggaggggtytttggttttttgaaacattgaaagcctttacaratgtgataagtgcagtgttcctgtgtgtctgcaccagaggcttctgwgacctgggaagagcattgtgattgagattcartgcttga

SEQ ID NO: 11 - DNA, 610 bp, human polyomavirus
aaatgtycctccagttcttcatataacaaacactgccacaacagtgctgcttgatgaatttggtgttgggccactttgcaaaggtgacaacttgtatttgtcagctgttgatgtttgtggcatgtttactaacagatctggttcccagcagtggagaggactgtccagatattttaaggttcagctcagaaaaagragggttaaaaacccctacccaatttctttccttcttactgatttaattaacagaaggacccctagagttgatggacagcctatgtatggsatggatgctcaggtagaggaggttagagtttttgaggggacagaggaacttccaggggacccagacatgatgagatatgttgacagatatggacagttgcagacaaaaaygctgtaatcaaaagcctttattgtaatatgcagtayattttaataaagtataaccagctttactttacagttgcagttattttgggggaggggtttttggttttttgaaacattggaagcctttacagatgtgataagtgcagtgttcctgtgtgtctgcaccagaggcttctgagacctgggaagagcattgtgattgagattcagtgcttga

SEQ ID NO: 12 - DNA, 610 bp, human polyomavirus
aaatgttcctccagttcttcatataacaaacactgccacaacagtgttgcttgatgaatttggtgttgggccactttgcaaaggtgacaacttgtatttgtcagctgttgatgtttgtggcatgtttactaacagatctggttcccagcagtggagaggactgtccagatattttaaggttcagctaagaaaaaggagggtgaaaaacccctacccaatttctttccttcttactgatttaattaacagaaggacccctagagttgatgggcagcctatgtatggcatggatgctcaggtagaggaggttagagtttttgagggsacagaggaacttccaggggacccagacatgatgagatatgttgacagatatggacagttgcagacaaagatgctgtaatcaaaagcctttattgtaatatgcagtacattttaataaagtatraccagcttcactttacagttgcagttattttgggggaggggtttttggttttttgaaacattgaaagcctttacagatgtgataggtgcagtgttcctgtgtgtctgcaccagaggcttctgagacctgggaaragcattgtgattgagattcagtgcttga

SEQ ID NO: 13 - DNA, 610 bp, human polyomavirus
aaatgttcctccagttcttcatatwacaaacactgccacaacagtgctgcttgatgaatttggtgttgggccactttgcaaaggtgacaacttgtatytgtcagctgttgatgtttgtggcatgtttactaacagatctggttcccagcartggagaggactgtccagatattttaaggttcarctaagaaaaaggagggttaaaaacccctacccaatttctttycttcttactgayttaattaacagaagracccctagagttgatgggcagcctatgtatggcatggatgctcaggtagaggaggttagagtktttgaggggacrgaggaacttccaggggacccagacatgatgagatatgttgacagatatggacarttgcagacaaagatgctgtaatcaaaggcctttattgtaataygcagtayattttaataaagtrtwaccagctttrcttwacwgttgcarttattttgggggaggggtttttggttttttgaaacattgraagcctttacagatgtgataagtgcagtgttcctgtgtgtctgcaccagaggcttctgagacctgggaaragcattgtgattgagattcagtgcttga

SEQ ID NO: 14 - DNA, 610 bp, human polyomavirus
aaatgttcctccagttcttcatataacmaacactgccacaacagtgctgcttgatgaatttggtgttggdccactttgcaaaggtgacaacttgtayttgtcagctgttgatgtttgtggcatgtttactaacagatctggttcccagcagtggagaggactrtccagatattttaaggtkcagctamgaaaraggagggttaaaaacccctacccaatttctttycttcttactgatttaattaacagaaggacccctagagttgatgggcagcctatgtatggcatggatgctcaggtagaggaggttagagtktttgagggracagaggaacttccaggggacccagacatgatgagatatgttgacaratatggacagttgcagacmaagatgctgtaatsaaargcctttattgtaatatgcartacattttaataaagtwtwaccagctktactkwacakttgcartyattttkggggaggggyttttggttttttgaarcattgaaagcctttacagatgtgataagtgcagtgttcctgtgtgtctgcaccagaggcttctgagacctgggaagagcattgtgattgagatymrkkscttga

SEQ ID NO: 15 - DNA, 610 bp, human polyomavirus
aaatgttcctccagttcttcatataacaaacactgccacaacagtgctgcttgatgaatttggtgtagggccactttgcaaaggtgacaacttgtatttgtcagctgttgatgtttgtggcatgtttactaacagatctggttcccagcagtggagaggactgtccagatattttaaggttcagctaaggaaaaggagggttaaaaacccctacccaatttctttccttcttactgatttaattaacagaaggactccaagagttgatgggcagcctatgtatggcatggatgctcaggtagaggaggttagagtttttgaggggacagaggaacttccaggggacccagacatgatgagatatgttgacagatatggacagttgcagacaaaaatgctgtaatcaaaagmctttattgtaatatgcagtacattttaataaagtataaccagctttactttacagttgcagttattttgggggaggggtttttggttttttgaaacattgaaagcctttacagatgtgataagtgcagtgttcctgtgtgtctgcaccagaggcttctgagacctgggaagagcattgtgattgagattcagtgcttga

SEQ ID NO: 16 - DNA, 610 bp, human polyomavirus
aaatgtkcctccagttcttcatatwacaaacactgccacwacagtgctrcttgatgaatttggtgttgggccactttgcaaaggtgacaacttgtatttgtcagctgttgatgtttgtggcatgtttacyaacagatctggktcccagcagtggagaggactctccagatattttaaggttcagctaagraaaaggagggttaaaaayccctacccaatttctttccttctgactgatttaatyaacagaaggactcctagagtwgatgggcagcctatgtatggcatggatgctcargtagaggaggtyagagtttttgaggggacagaggaacttccaggggacccagacatgatgasatatgttgacagatatggacagttgcrgaccaaaatgctgtaatcataagcctttattgtaatatgcagtacattttaataaagtataaccagctttactytacagttkcacttattttgggggagggktttttggtttyttraaacattgaaagcctttacagatgtgataagtgcagkgttcctgtgtgtctgcaccagaggcttctgagacctgggaagagcattgtgattgagattcagtgcttga

SEQ ID NO: 17 - DNA, 20 bp, Artificial Sequence
  Synthetic DNA
acaacagtgctgcttgatga

SEQ ID NO: 18 - DNA, 20 bp, Artificial Sequence
  Synthetic DNA
acaacagtgttgcttgatga

SEQ ID NO: 19 - DNA, 20 bp, Artificial Sequence
  Synthetic DNA
acaacagtgttgctcgatga

SEQ ID NO: 20 - DNA, 20 bp, Artificial Sequence
  Synthetic DNA
aatttggtgttgggccactt

SEQ ID NO: 21 - DNA, 20 bp, Artificial Sequence
  Synthetic DNA
aatttggtgtagggccactt

SEQ ID NO: 22 - DNA, 20 bp, Artificial Sequence
  Synthetic DNA
aatttggtgttgggcctctt

SEQ ID NO: 23 - DNA, 20 bp, Artificial Sequence
  Synthetic DNA
gtcagctgttgatgtttgtg

SEQ ID NO: 24 - DNA, 20 bp, Artificial Sequence
  Synthetic DNA
gtcagctgtggatgtttgtg

SEQ ID NO: 25 - DNA, 20 bp, Artificial Sequence
  Synthetic DNA
cagctaagaaaaaggagggt

SEQ ID NO: 26 - DNA, 20 bp, Artificial Sequence
  Synthetic DNA
cagctaaggaaaaggagggt

SEQ ID NO: 27 - DNA, 20 bp, Artificial Sequence
  Synthetic DNA
cagctgagaaaaaggagggt

SEQ ID NO: 28 - DNA, 20 bp, Artificial Sequence
  Synthetic DNA
aaaggagggttaaaaacccc

SEQ ID NO: 29 - DNA, 20 bp, Artificial Sequence
  Synthetic DNA
aaaggagggtgaaaaacccc

SEQ ID NO: 30 - DNA, 20 bp, Artificial Sequence
  Synthetic DNA
aaaggagggtcaagaacccc

SEQ ID NO: 31 - DNA, 20 bp, Artificial Sequence
  Synthetic DNA
aaaggagggttaagaacccc

SEQ ID NO: 32 - DNA, 20 bp, Artificial Sequence
  Synthetic DNA
ctttccttcttactgattta

SEQ ID NO: 33 - DNA, 20 bp, Artificial Sequence
  Synthetic DNA
ctttccttctgactgattta

SEQ ID NO: 34 - DNA, 20 bp, Artificial Sequence
  Synthetic DNA
cttactgatttaattaacag

SEQ ID NO: 35 - DNA, 20 bp, Artificial Sequence
  Synthetic DNA
cttactgatttgattaacag

SEQ ID NO: 36 - DNA, 20 bp, Artificial Sequence
  Synthetic DNA
agaaggacccctagagttga

SEQ ID NO: 37 - DNA, 20 bp, Artificial Sequence
  Synthetic DNA
agaaggaccccaagagttga

SEQ ID NO: 38 - DNA, 20 bp, Artificial Sequence
  Synthetic DNA
agaaggactcctagagtaga

SEQ ID NO: 39 - DNA, 20 bp, Artificial Sequence
  Synthetic DNA
agaaggactcctagagttga

SEQ ID NO: 40 - DNA, 20 bp, Artificial Sequence
  Synthetic DNA
gagttgatgggcagcctatg

SEQ ID NO: 41 - DNA, 20 bp, Artificial Sequence
  Synthetic DNA
gagttgatggacagcctatg

SEQ ID NO: 42 - DNA, 20 bp, Artificial Sequence
  Synthetic DNA
ctatgtatggcatggatgct

SEQ ID NO: 43 - DNA, 20 bp, Artificial Sequence
  Synthetic DNA
ctatgtatggtatggatgct

SEQ ID NO: 44 - DNA, 20 bp, Artificial Sequence
  Synthetic DNA
ctatgtatggtatggacgct

SEQ ID NO: 45 - DNA, 20 bp, Artificial Sequence
  Synthetic DNA
ctatgtacggtatggatgct

SEQ ID NO: 46 - DNA, 20 bp, Artificial Sequence
  Synthetic DNA
tggatgctcaggtagaggag

SEQ ID NO: 47 - DNA, 20 bp, Artificial Sequence
  Synthetic DNA
tggatgctcaaatagaggag

SEQ ID NO: 48 - DNA, 20 bp, Artificial Sequence
  Synthetic DNA
tggatgctcagatagaggag

SEQ ID NO: 49 - DNA, 20 bp, Artificial Sequence
  Synthetic DNA
ttgaggggacagaggaactt

SEQ ID NO: 50 - DNA, 20 bp, Artificial Sequence
  Synthetic DNA
ttgagggaacagaggagctt

SEQ ID NO: 51 - DNA, 20 bp, Artificial Sequence
  Synthetic DNA
ttgaggggacagagcaactt

SEQ ID NO: 52 - DNA, 20 bp, Artificial Sequence
  Synthetic DNA
gggacccagacatgatgaga

SEQ ID NO: 53 - DNA, 20 bp, Artificial Sequence
  Synthetic DNA
gggacccagatatgatgaga

SEQ ID NO: 54 - DNA, 20 bp, Artificial Sequence
  Synthetic DNA
agttgcagacaaagatgctg

SEQ ID NO: 55 - DNA, 20 bp, Artificial Sequence
  Synthetic DNA
agttgcaaacaaagatgctg

SEQ ID NO: 56 - DNA, 20 bp, Artificial Sequence
  Synthetic DNA
agttgcagacaaaaatgctg

SEQ ID NO: 57 - DNA, 20 bp, Artificial Sequence
  Synthetic DNA
gctgtaatcaaaagccttta

SEQ ID NO: 58 - DNA, 20 bp, Artificial Sequence
  Synthetic DNA
gctgtaatgaaaagccttta

SEQ ID NO: 59 - DNA, 20 bp, Artificial Sequence
  Synthetic DNA
gctgtaatcaaaggccttta

SEQ ID NO: 60 - DNA, 20 bp, Artificial Sequence
  Synthetic DNA
gctgtaatcataagccttta

SEQ ID NO: 61 - DNA, 20 bp, Artificial Sequence
  Synthetic DNA
aataaagtataaccagcttt

SEQ ID NO: 62 - DNA, 20 bp, Artificial Sequence
  Synthetic DNA
aataaagtatgaccagcttc

SEQ ID NO: 63 - DNA, 20 bp, Artificial Sequence
  Synthetic DNA
ataaccagctttactttaca

SEQ ID NO: 64 - DNA, 20 bp, Artificial Sequence
  Synthetic DNA
ataaccagctgtacttaaca

SEQ ID NO: 65 - DNA, 20 bp, Artificial Sequence
  Synthetic DNA
ttaaccagctgtacttaaca

SEQ ID NO: 66 - DNA, 20 bp, Artificial Sequence
  Synthetic DNA
cagctttactttacagttgc

SEQ ID NO: 67 - DNA, 20 bp, Artificial Sequence
  Synthetic DNA
cagcttcacttgacagttgc

SEQ ID NO: 68 - DNA, 20 bp, Artificial Sequence
  Synthetic DNA
cagctttacttgacagttgc

SEQ ID NO: 69 - DNA, 20 bp, Artificial Sequence
  Synthetic DNA
gaaacattgaaagcctttac

SEQ ID NO: 70 - DNA, 20 bp, Artificial Sequence
  Synthetic DNA
gaaacattggaagcctttac

SEQ ID NO: 71 - DNA, 29 bp, Artificial Sequence
  Synthetic DNA
cacaagcttttttggacactaacaggagg

SEQ ID NO: 72 - DNA, 26 bp, Artificial Sequence
  Synthetic DNA
gattctgcagaagactctggacatgg

(19) United States
(12) Patent Application Publication
(10) Pub. No.: US 2008/0188376 A1
(43) Pub. Date: Aug. 7, 2008

(54) Oligonucleotide probes and microarray for determining genome types

(75) Inventors: Hiroshi Ikegaya, Chiba (JP); Koichi Sakurada, Chiba (JP); Koichi Hirayama, Yamaguchi (JP); Kenji Isogai, Tokyo (JP)

Correspondence Address: BIRCH STEWART KOLASCH & BIRCH, PO BOX 747, FALLS CHURCH, VA, 22040-0747, US

(73) Assignees: Toyo Kohan Co., Ltd., Tokyo (JP); National Research Institute of Police Science, Kashiwa-shi (JP)

(21) Appl. No.: 11/987,919

(22) Filed: Dec. 5, 2007

(30) Foreign Application Priority Data
Dec. 6, 2006 (JP) 2006-329735

(51) Int. Cl. C40B 30/04; C07H 21/00; C40B 40/08; C12Q 1/70

(52) U.S. Cl. 506/9; 536/24.32; 506/17; 435/5

(57) ABSTRACT

According to the present invention, a means of estimating the place of origin of a test subject by quickly and conveniently determining the genome type of JC virus infecting a test subject is provided. The present invention relates to a set of oligonucleotide probes for determining the genome type of JC virus infecting a test subject, such set containing: oligonucleotide probes (a-1) and (a-2); oligonucleotide probes (b-1) and (b-2); oligonucleotide probes (c-1) and (c-2); oligonucleotide probes (d-1), (d-2), (d-3), and (d-4); oligonucleotide probes (e-1), (e-2), and (e-3); oligonucleotide probes (f-1), (f-1), and (f-3); oligonucleotide probes (g-1), (g-2), and (g-3); oligonucleotide probes (h-1), (h-2), (h-3), and (h-4); oligonucleotide probes (i-1) and (i-2); oligonucleotide probes (j-1), (j-2), and (j-3); oligonucleotide probes (k-1), (k-2), and (k-3); and oligonucleotide probes (l-1) and (l-2).

### BACKGROUND OF THE INVENTION

1. Field of the Invention

The present invention relates to oligonucleotide probes and a microarray for determining the genome type of JC virus infecting a test subject. The present invention also relates to a method for determining the genome type of JC virus infecting a test subject and a method for determining the place of origin of the test subject with the use of the probes and the microarray.

2. Background Art

As a result of world-wide serological research, it was revealed that JC virus (JCV), which is a kind of Papovavirus, has spread throughout human populations, and that most people are asymptomatically infected with JCV in their childhood. Complete removal of JCV in vivo by an immunological reaction is impossible. Some JC viruses reach kidney tissue, peripheral lymphocytes, and lymphoid tissue, and such tissues and lymphocytes are persistently infected through life. In adults, JCVs in kidney tissue actively proliferate, and proliferated JC viruses are excreted in urine. JCVs excreted in urine invade uninfected children, resulting in the induction of new infection. From the earliest time of the dawn of humanity, JCVs have repeated such infection cycle and have lived with humans.

In order to clarify the origin of JCV, DNA analysis of JCV genomes obtained by cloning of the genomes derived from various ethnic groups throughout the world was conducted, during which it was revealed that JCV genome types relate to specific races. Hitherto, the nucleotide sequences of the IG regions (610 base pairs) of JCV genomic DNAs have been determined with the use of urine collected from all over the world. Then, a molecular phylogenetic tree was established based on the obtained nucleotide sequences by the neighbor-joining method (NJ method). According to the phylogenetic tree, it was revealed that JCVs throughout the world can be divided into 12 types (genome types). Each genome type has a specific distribution area. For instance, the genome type EU is distributed throughout the whole of Europe and the Mediterranean region. In addition, the genome type Af2 is distributed throughout the whole of Africa and West Asia (including India). Such findings indicate that JCV genome types closely relate to human populations. Thus, JCV genome types have been gaining attention as a new index for human populations. In addition, it has been reported that a JCV genome type detected from human urine or kidney can be used in connection with a method to estimate the place of origin of an unidentified cadaver.

Hitherto, in order to accurately determine a JCV genome type, a method comprising determining the nucleotide sequence of a genome type and carrying out molecular phylogenetic analysis has been used (JOURNAL OF CLINICAL MICROBIOLOGY, June 1995, pp. 1448-1451). However, such conventional method requires professional analytical skills and is time-consuming, which have been problematic. Also, such method cannot be applied to, for example, a minute amount of a sample.

### SUMMARY OF THE INVENTION

It is an objective of the present invention to provide a means of estimating the place of origin of a test subject by quickly and conveniently determining the genome type of JC virus infecting the test subject.

The present inventors succeeded in designing a set of oligonucleotide probes that are useful for determination of JC virus genome types based on the nucleotide sequences of genomic DNAs of JC viruses. This has led to the completion of the present invention.

Specifically, the present invention encompasses the following inventions.
(1) A set of oligonucleotide probes for determining the genome type of JC virus infecting a test subject, wherein the set containing: oligonucleotide probes (a-1) and (a-2); oligonucleotide probes (b-1) and (b-2); oligonucleotide probes (c-1) and (c-2); oligonucleotide probes (d-1), (d-2), (d-3), and (d-4); oligonucleotide probes (e-1), (e-2), and (e-3); oligonucleotide probes (f-1), (f-2), and (f-3); oligonucleotide probes (g-1), (g-2), and (g-3); oligonucleotide probes (h-1), (h-2), (h-3), and (h-4); oligonucleotide probes (i-1) and (i-2); oligonucleotide probes (j-1), (j-2), and (j-3); oligonucleotide probes (k-1), (k-2), and (k-3); and oligonucleotide probes (l-1) and (l-2).
(2) A microarray for determining the genome type of JC virus infecting a test subject, wherein the set of oligonucleotide probes according to (1) is immobilized on a carrier.
(3) A microarray for assuming a place of origin of a test subject, wherein the set of oligonucleotide probes according to (1) is immobilized on a carrier.
(4) The microarray according to (2) or (3), wherein the carrier has a carbon layer and a chemically modifying group on the surface thereof.
(5) A method for determining the genome type of JC virus infecting a test subject, comprising the steps of:

extracting DNA from a sample derived from a test subject,

amplifying the nucleic acid encoding the IG region of a JC virus genome with the use of the extracted DNA as a template; and

detecting the amplified nucleic acid with the use of the set of oligonucleotide probes according to (1) or the microarray according to (2) to (4).
(6) A method for estimating a place of origin of a test subject based on the JC virus genome type determined by the method according to (5).

According to the present invention, it becomes possible to quickly and conveniently determine the genome type of JC virus infecting a test subject with the use of a minute amount of a sample. In addition, according to the present invention, a means whereby persons who do not have highly professional knowledge can determine the genome type of JC virus is provided. Further, it is also possible to estimate a place of origin of a test subject based on the genome type of JC virus.

### BRIEF DESCRIPTION OF THE DRAWINGS

FIG. 1-1 shows 54 types of oligonucleotide probes designed based on the nucleotide sequences (SEQ ID NO: 1 to 16) of 16 different genome types of JC virus.

FIG. 1-2 shows 54 types of oligonucleotide probes designed based on the nucleotide sequences (SEQ ID NO: 1 to 16) of 16 different genome types of JC virus.

FIG. 1-3 shows 54 types of oligonucleotide probes designed based on the nucleotide sequences (SEQ ID NO: 1 to 16) of 16 different genome types of JC virus.

FIG. 2 shows xy plotting results for fluorescence intensities obtained in two tests arbitrarily selected from among five tests with the use of identical samples in Example 3.

FIG. 3 shows signal intensities (corresponding to probes) obtained from samples 1 to 11 in Example 4. In each probe group, a value corresponding to the strongest signal intensity obtained with the relevant probe is shown in bold. Also, a value corresponding to a signal characteristic to either one of the genome types is shown in hatching. The determination results are shown in the bottom row.

### DESCRIPTION OF THE PREFERRED EMBODIMENTS

JC virus belongs to the Polyomavirus family, and the host thereof is a human. The genome of JC virus is circular double-strand DNA approximately 5100 base pairs in length. The set of oligonucleotide probes of the present invention is used for detection of the IG region of the genome type of JC virus infecting a test subject. It has been reported that the IG region is a 610-base-pair region with frequent mutations in the genome of JC virus (J Gen Virol, 73:2669-2678, 1992) and the genomes of the region can be classified into 12 types (Proc Natl Acad Sci USA, 94: 9191-9196, 1997; J Gen Virol, 79: 2499-2505, 1998). Genomes of the IG region are classified into the following types: the EU-a type, the EU-b type, the Af1 type, the Af2 type, the SC type, the CY type, the MY type, the B1-a type, the B1-b type, the B1-c type, the B1-d type, and the B2 type.

According to the present invention, determination of the genome type of JC virus infecting a test subject includes determination of the genome type of JC virus infecting a test subject as the origin of a test sample. Also, estimating the place of origin of a test subject includes estimation of the place of origin of a test subject as the origin of a test sample.

The set of oligonucleotide probes of the present invention contains at least oligonucleotide probe groups (a) to (1): an oligonucleotide probe group (a) comprising oligonucleotide probes (a-1) and (a-2); an oligonucleotide probe group (b) comprising oligonucleotide probes (b-1) and (b-2); an oligonucleotide probe group (c) comprising oligonucleotide probes (c-1) and (c-2); an oligonucleotide probe group (d) comprising oligonucleotide probes (d-1), (d-2), (d-3), and (d-4); an oligonucleotide probe group (e) comprising oligonucleotide probes (e-1), (e-2), and (e-3); an oligonucleotide probe group (f) comprising oligonucleotide probes (f-1), (f-2), and (f-3); an oligonucleotide probe group (g) comprising oligonucleotide probes (g-1), (g-2), and (g-3); an oligonucleotide probe group (h) comprising oligonucleotide probes (h-1), (h-2), (h-3), and (h-4); an oligonucleotide probe group (i) comprising oligonucleotide probes (i-1) and (i-2); an oligonucleotide probe group (j) comprising oligonucleotide probes (j-1), (j-2), and (j-3); an oligonucleotide probe group (k) comprising oligonucleotide probes (k-1), (k-2), and (k-3); and an oligonucleotide probe group (l) comprising oligonucleotide probes (l-1) and (l-2).

The set of oligonucleotide probes of the present invention may further contain oligonucleotide probe groups (m) to (s): an oligonucleotide probe group (m) comprising oligonucleotide probes (m-1), (m-2), and (m-3); an oligonucleotide probe group (n) comprising oligonucleotide probes (n-1), (n-2), and (n-3); an oligonucleotide probe group (o) comprising oligonucleotide probes (o-1), (o-2), and (o-3); an oligonucleotide probe group (p) comprising oligonucleotide probes (p-1), (p-2), (p-3), and (p-4); an oligonucleotide probe group (q) comprising oligonucleotide probes (q-1) and (q-2); an oligonucleotide probe group (r) comprising oligonucleotide probes (r-1), (r-2), (r-3), and (r-4); and an oligonucleotide probe group (s) comprising oligonucleotide probes (s-1) and (s-2).

The set of oligonucleotide probes of the present invention may contain a single type of oligonucleotide probe group or plural types of oligonucleotide probe groups as each of the the oligonucleotide probe group (such as an oligonucleotide probe group (a)), as long as it complies with required conditions. Likewise, in the set of oligonucleotide probes of the present invention, each oligonucleotide probe group may comprise a single type of oligonucleotide probe or plural types of oligonucleotide probes as each of the oligonucleotide probe (such as an oligonucleotide probe (a-1)), as long as it complies with required conditions.

Hereinafter, oligonucleotide probes contained in each oligonucleotide probe group are explained.

### Oligonucleotide Probe Group (a):

An oligonucleotide probe (a-1) is an oligonucleotide probe having a nucleotide sequence of 10 to 30 consecutive nucleotides including nucleotides at positions 100 to 119 of SEQ ID NO: 2, 4, 5, 6, 7, 9, 10, 11, 12, 13, 14, 15, or 16 (preferably including the nucleotides represented by SEQ ID NO: 23). The probe is preferably an oligonucleotide probe having the nucleotide sequence represented by SEQ ID NO: 23. An oligonucleotide probe (a-2) is an oligonucleotide probe having a nucleotide sequence of 10 to 30 consecutive nucleotides including nucleotides at positions 100 to 119 of SEQ ID NO: 3 (preferably including the nucleotides represented by SEQ ID NO: 24). The probe is preferably an oligonucleotide probe having the nucleotide sequence represented by SEQ ID NO: 24.

### Oligonucleotide Probe Group (b):

An oligonucleotide probe (b-1) is an oligonucleotide probe having a nucleotide sequence of 10 to 30 consecutive nucleotides including nucleotides at positions 222 to 241 of SEQ ID NO: 11, 12, 14, or 15 (preferably including the nucleotides represented by SEQ ID NO: 32). The probe is preferably an oligonucleotide probe having the nucleotide sequence represented by SEQ ID NO: 32. An oligonucleotide probe (b-2) is an oligonucleotide probe having a nucleotide sequence of 10 to 30 consecutive nucleotides including nucleotides at positions 222 to 241 of SEQ ID NO: 16 (preferably including the nucleotides represented by SEQ ID NO: 33). The probe is preferably an oligonucleotide probe having the nucleotide sequence represented by SEQ ID NO: 33.

### Oligonucleotide Probe Group (c):

An oligonucleotide probe (c-1) is an oligonucleotide probe having a nucleotide sequence of 10 to 30 consecutive nucleotides including nucleotides at positions 261 to 280 of SEQ ID NO: 1, 3, 4, 6, 9, 12, 13, 14, or 15 (preferably including the nucleotides represented by SEQ ID NO: 40). The probe is preferably an oligonucleotide probe having the nucleotide sequence represented by SEQ ID NO: 40. An oligonucleotide probe (c-2) is an oligonucleotide probe having a nucleotide sequence of 10 to 30 consecutive nucleotides including nucleotides at positions 261 to 280 of SEQ ID NO: 11 (preferably including the nucleotides represented by SEQ ID NO: 41). The probe is preferably an oligonucleotide probe having the nucleotide sequence represented by SEQ ID NO: 41.

### Oligonucleotide Probe Group (d):

An oligonucleotide probe (d-1) is an oligonucleotide probe having a nucleotide sequence of 10 to 30 consecutive nucleotides including nucleotides at positions 276 to 295 of SEQ ID NO: 1, 3, 6, 12, 13, or 14 (preferably including the nucleotides represented by SEQ ID NO: 42). The probe is preferably an oligonucleotide probe having the nucleotide sequence represented by SEQ ID NO: 42. An oligonucleotide probe (d-2) is an oligonucleotide probe having a nucleotide sequence of 10 to 30 consecutive nucleotides including nucleotides at positions 276 to 295 of SEQ ID NO: 4, 7, 9, 10, 15, or 16 (preferably including the nucleotides represented by SEQ ID NO: 43). The probe is preferably an oligonucleotide probe having the nucleotide sequence represented by SEQ ID NO: 43. An oligonucleotide probe (d-3) is an oligonucleotide probe having a nucleotide sequence of 10 to 30 consecutive nucleotides including nucleotides at positions 276 to 295 of SEQ ID NO: 4, 7, 9, 10, 15, or 16 in which T at position 292 is substituted with C (preferably including the nucleotides represented by SEQ ID NO: 44). The probe is preferably an oligonucleotide probe having the nucleotide sequence represented by SEQ ID NO: 44. An oligonucleotide probe (d-4) is an oligonucleotide probe having a nucleotide sequence of 10 to 30 consecutive nucleotides including nucleotides at positions 276 to 295 of SEQ ID NO: 4, 7, 9, 10, 15, or 16 in which T at position 283 is substituted with C (preferably including the nucleotides represented by SEQ ID NO: 45). The probe is preferably an oligonucleotide probe having the nucleotide sequence represented by SEQ ID NO: 45.

### Oligonucleotide Probe Group (e):

An oligonucleotide probe (e-1) is an oligonucleotide probe having a nucleotide sequence of 10 to 30 consecutive nucleotides including nucleotides at positions 288 to 307 of SEQ ID NO: 3, 4, 5, 6, 8, 9, 10, 11, 12, 13, 14, or 15 (preferably including the nucleotides represented by SEQ ID NO: 46). The probe is preferably an oligonucleotide probe having the nucleotide sequence represented by SEQ ID NO: 46. An oligonucleotide probes (e-2) is an oligonucleotide probe having a nucleotide sequence of 10 to 30 consecutive nucleotides including nucleotides at positions 288 to 307 of SEQ ID NO: 3, 4, 5, 6, 8, 9, 10, 11, 12, 13, 14, or 15 in which Gs at positions 298 and 299 are substituted with A (preferably including the nucleotides represented by SEQ ID NO: 47). The probe is preferably an oligonucleotide probe having the nucleotide sequence represented by SEQ ID NO: 47. An oligonucleotide probes (e-3) is an oligonucleotide probe having a nucleotide sequence of 10 to 30 consecutive nucleotides including nucleotides at positions 288 to 307 of SEQ ID NO: 3, 4, 5, 6, 8, 9, 10, 11, 12, 13, 14, or 15 in which G at position 299 is substituted with A (preferably including the nucleotides represented by SEQ ID NO: 48). The probe is preferably an oligonucleotide probe having the nucleotide sequence represented by SEQ ID NO: 48.

### Oligonucleotide Probe Group (f):

An oligonucleotide probes (f-1) is an oligonucleotide probe having a nucleotide sequence of 10 to 30 consecutive nucleotides including nucleotides at positions 318 to 337 of SEQ ID NO: 3, 4, 5, 8, 9, 10, 11, 12, 13, 15, or 16 (preferably including the nucleotides represented by SEQ ID NO: 49). The probe is preferably an oligonucleotide probe having the nucleotide sequence represented by SEQ ID NO: 49. An oligonucleotide probe (f-2) is an oligonucleotide probe having a nucleotide sequence of 10 to 30 consecutive nucleotides including nucleotides at positions 318 to 337 of SEQ ID NO: 1 (preferably including the nucleotides represented by SEQ ID NO: 50). The probe is preferably an oligonucleotide probe having the nucleotide sequence represented by SEQ ID NO: 50. An oligonucleotide probe (f-3) is an oligonucleotide probe having a nucleotide sequence of 10 to 30 consecutive nucleotides including nucleotides at positions 318 to 337 of SEQ ID NO: 2 (preferably including the nucleotides represented by SEQ ID NO: 51). The probe is preferably an oligonucleotide probe having the nucleotide sequence represented by SEQ ID NO: 51.

### Oligonucleotide Probe Group (g):

An oligonucleotide probes (g-1) is an oligonucleotide probe having a nucleotide sequence of 10 to 30 consecutive nucleotides including nucleotides at positions 381 to 400 of SEQ ID NO: 12, 13, or 14 (preferably including the nucleotides represented by SEQ ID NO: 54). The probe is preferably an oligonucleotide probe having the nucleotide sequence represented by SEQ ID NO: 54. An oligonucleotide probe (g-2) is an oligonucleotide probe having a nucleotide sequence of 10 to 30 consecutive nucleotides including nucleotides at positions 381 to 400 of SEQ ID NO: 4, 5, 9, or 10 (preferably including the nucleotides represented by SEQ ID NO: 55). The probe is preferably an oligonucleotide probe having the nucleotide sequence represented by SEQ ID NO: 55. An oligonucleotide probe (g-3) is an oligonucleotide probe having a nucleotide sequence of 10 to 30 consecutive nucleotides including nucleotides at positions 381 to 400 of SEQ ID NO: 15 (including nucleotides represented by SEQ ID NO: 56). The probe is preferably an oligonucleotide probe having the nucleotide sequence represented by SEQ ID NO: 56.

### Oligonucleotide Probe Group (h):

An oligonucleotide probe (h-1) is an oligonucleotide probe having a nucleotide sequence of 10 to 30 consecutive nucleotides including nucleotides at positions 397 to 416 of SEQ ID NO: 5, 9, 11, or 12 (preferably including the nucleotides represented by SEQ ID NO: 57). The probe is preferably an oligonucleotide probe having the nucleotide sequence represented by SEQ ID NO: 57. An oligonucleotide probe (h-2) is an oligonucleotide probe having a nucleotide sequence of 10 to 30 consecutive nucleotides including nucleotides at positions 397 to 416 of SEQ ID NO: 14 (preferably including the nucleotides represented by SEQ ID NO: 58). The probe is preferably an oligonucleotide probe having the nucleotide sequence represented by SEQ ID NO: 58. An oligonucleotide probe (h-3) is an oligonucleotide probe having a nucleotide sequence of 10 to 30 consecutive nucleotides including nucleotides at positions 397 to 416 of SEQ ID NO: 13 (preferably including the nucleotides represented by SEQ ID NO: 59). The probe is preferably an oligonucleotide probe having the nucleotide sequence represented by SEQ ID NO: 59. An oligonucleotide probes (h-4) is an oligonucleotide probe having a nucleotide sequence of 10 to 30 consecutive nucleotides including nucleotides at positions 397 to 416 of SEQ ID NO: 16 (preferably including the nucleotides represented by SEQ ID NO: 60). The probe is preferably an oligonucleotide probe having the nucleotide sequence represented by SEQ ID NO: 60.

### Oligonucleotide Probe Group (i):

An oligonucleotide probe (i-1) is an oligonucleotide probe having a nucleotide sequence of 10 to 30 consecutive nucleotides including nucleotides at positions 438 to 457 of SEQ ID NO: 5, 11, 13, 15, or 16 (preferably including the nucleotides represented by SEQ ID NO: 61). The probe is preferably an oligonucleotide probe having the nucleotide sequence represented by SEQ ID NO: 61. An oligonucleotide probes (i-2) is an oligonucleotide probe having a nucleotide sequence of 10 to 30 consecutive nucleotides including nucleotides at positions 438 to 457 of SEQ ID NO: 12 (preferably including the nucleotides represented by SEQ ID NO: 62). The probe is preferably an oligonucleotide probe having the nucleotide sequence represented by SEQ ID NO: 62.

### Oligonucleotide Probe Group (j):

An oligonucleotide probe (j-1) is an oligonucleotide probe having a nucleotide sequence of 10 to 30 consecutive nucleotides including nucleotides at positions 446 to 465 of SEQ ID NO: 5, 11, or 15 (preferably including the nucleotides represented by SEQ ID NO: 63). The probe is preferably an oligonucleotide probe having the nucleotide sequence represented by SEQ ID NO: 63. An oligonucleotide probe (j-2) is an oligonucleotide probe having a nucleotide sequence of 10 to 30 consecutive nucleotides including nucleotides at positions 446 to 465 of SEQ ID NO: 5, 11, or 15 in which T at position 456 is substituted with G and T at position 462 is substituted with A (preferably including the nucleotides represented by SEQ ID NO: 64). The probe is preferably an oligonucleotide probe having the nucleotide sequence represented by SEQ ID NO: 64. An oligonucleotide probes (j-3) is an oligonucleotide probe having a nucleotide sequence of 10 to 30 consecutive nucleotides including nucleotides at positions 446 to 465 of SEQ ID NO: 5, 11, or 15 in which A at position 446 is substituted with T, T at position 456 is substituted with G, and T at position 462 is substituted with A (preferably including the nucleotides represented by SEQ ID NO: 65). The probe is preferably an oligonucleotide probe having the nucleotide sequence represented by SEQ ID NO: 65.

### Oligonucleotide Probe Group (k):

An oligonucleotide probe (k-1) is an oligonucleotide probe having a nucleotide sequence of 10 to 30 consecutive nucleotides including nucleotides at positions 451 to 470 of SEQ ID NO: 5, 9, 11, or 15 (preferably including the nucleotides represented by SEQ ID NO: 66). The probe is preferably an oligonucleotide probe having the nucleotide sequence represented by SEQ ID NO: 66. An oligonucleotide probe (k-2) is an oligonucleotide probe having a nucleotide sequence of 10 to 30 consecutive nucleotides including nucleotides at positions 451 to 470 of SEQ ID NO: 5, 9, 11, or 15 in which T at position 457 is substituted with C and T at position 462 is substituted with G (preferably including the nucleotides represented by SEQ ID NO: 67). The probe is preferably an oligonucleotide probe having the nucleotide sequence represented by SEQ ID NO: 67. An oligonucleotide probe (k-3) is an oligonucleotide probe having a nucleotide sequence of 10 to 30 consecutive nucleotides including nucleotides at positions 451 to 470 of SEQ ID NO: 5, 9, 11, or 15 n which T at position 462 is substituted with G (preferably including the nucleotides represented by SEQ ID NO: 68). The probe is preferably an oligonucleotide probe having the nucleotide sequence represented by SEQ ID NO: 68.

### Oligonucleotide Probe Group (l):

An oligonucleotide probe (l-1) is an oligonucleotide probe having a nucleotide sequence of 10 to 30 consecutive nucleotides including nucleotides at positions 503 to 522 of SEQ ID NO: 3, 4, 5, 6, 7, 8, 9, 10, 11, 12, 13, 14, or 15 (preferably including the nucleotides represented by SEQ ID NO: 69). The probe is preferably an oligonucleotide probe having the nucleotide sequence represented by SEQ ID NO: 69. An oligonucleotide probe (l-2) is an oligonucleotide probe having a nucleotide sequence of 10 to 30 consecutive nucleotides including nucleotides at positions 503 to 522 of SEQ ID NO: 11 (preferably including the nucleotides represented by SEQ ID NO: 70). The probe is preferably an oligonucleotide probe having the nucleotide sequence represented by SEQ ID NO: 70.

### Oligonucleotide Probe Group (m):

An oligonucleotide probe (m-1) is an oligonucleotide probe having a nucleotide sequence of 10 to 30 consecutive nucleotides including nucleotides at positions 38 to 57 of SEQ ID NO: 2, 3, 5, 6, 9, 10, 11, 13, 14, or 15 (preferably including the nucleotides represented by SEQ ID NO: 17). The probe is preferably an oligonucleotide probe having the nucleotide sequence represented by SEQ ID NO: 17. An oligonucleotide probe (m-2) is an oligonucleotide probe having a nucleotide sequence of 10 to 30 consecutive nucleotides including nucleotides at positions 38 to 57 of SEQ ID NO: 12 (preferably including the nucleotides represented by SEQ ID NO: 18). The probe is preferably an oligonucleotide probe having the nucleotide sequence represented by SEQ ID NO: 18. An oligonucleotide probes (m-3) is an oligonucleotide probe having a nucleotide sequence of 10 to 30 consecutive nucleotides including nucleotides at positions 38 to 57 of SEQ ID NO: 12 in which T at position 52 is substituted with C (preferably including the nucleotides represented by SEQ ID NO: 19). The probe is preferably an oligonucleotide probe having the nucleotide sequence represented by SEQ ID NO: 19.

### Oligonucleotide Probe Group (n):

An oligonucleotide probes (n-1) is an oligonucleotide probe having a nucleotide sequence of 10 to 30 consecutive nucleotides including nucleotides at positions 57 to 76 of SEQ ID NO: 3, 6, 9, 11, 12, 13, or 16 (preferably including the nucleotides represented by SEQ ID NO: 20). The probe is preferably an oligonucleotide probe having the nucleotide sequence represented by SEQ ID NO: 20. An oligonucleotide probe (n-2) is an oligonucleotide probe having a nucleotide sequence of 10 to 30 consecutive nucleotides including nucleotides at positions 57 to 76 of SEQ ID NO: 15 (preferably including the nucleotides represented by SEQ ID NO: 21). The probe is preferably an oligonucleotide probe having the nucleotide sequence represented by SEQ ID NO: 21. An oligonucleotide probe (n-3) is an oligonucleotide probe having a nucleotide sequence of 10 to 30 consecutive nucleotides including nucleotides at positions 57 to 76 of SEQ ID NO: 4 or 5 (preferably including the nucleotides represented by SEQ ID NO: 22). The probe is preferably an oligonucleotide probe having the nucleotide sequence represented by SEQ ID NO: 22.

### Oligonucleotide Probe Group (o):

An oligonucleotide probe (o-1) is an oligonucleotide probe having a nucleotide sequence of 10 to 30 consecutive nucleotides including nucleotides at positions 182 to 201 of SEQ ID NO: 4, 6, 12, 13, or 14 (preferably including the nucleotides represented by SEQ ID NO: 25). The probe is preferably an oligonucleotide probe having the nucleotide sequence represented by SEQ ID NO: 25. An oligonucleotide probe (o-2) is an oligonucleotide probe having a nucleotide sequence of 10 to 30 consecutive nucleotides including nucleotides at positions 182 to 201 of SEQ ID NO: 15 (preferably including the nucleotides represented by SEQ ID NO: 26). The probe is preferably an oligonucleotide probe having the nucleotide sequence represented by SEQ ID NO: 26. An oligonucleotide probe (o-3) is an oligonucleotide probe having a nucleotide sequence of 10 to 30 consecutive nucleotides including nucleotides at positions 182 to 201 of SEQ ID NO: 5 or 10 (preferably including the nucleotides represented by SEQ ID NO: 27). The probe is preferably an oligonucleotide probe having the nucleotide sequence represented by SEQ ID NO: 27.

### Oligonucleotide Probe Group (p):

An oligonucleotide probe (p-1) is an oligonucleotide probe having a nucleotide sequence of 10 to 30 consecutive nucleotides including nucleotides at positions 192 to 211 of SEQ ID NO: 5, 6, 10, 13, 14, or 15 (preferably including the nucleotides represented by SEQ ID NO: 28). The probe is preferably an oligonucleotide probe having the nucleotide sequence represented by SEQ ID NO: 28. An oligonucleotide probe (p-2) is an oligonucleotide probe having a nucleotide sequence of 10 to 30 consecutive nucleotides including nucleotides at positions 192 to 211 of SEQ ID NO: 12 (preferably including the nucleotides represented by SEQ ID NO: 29). The probe is preferably an oligonucleotide probe having the nucleotide sequence represented by SEQ ID NO: 29. An oligonucleotide probe (p-3) is an oligonucleotide probe having a nucleotide sequence of 10 to 30 consecutive nucleotides including nucleotides at positions 192 to 211 of SEQ ID NO: 12 in which G at position 102 is substituted with C and A at position 105 is substituted with & (preferably including the nucleotides represented by SEQ ID NO: 30). The probe is preferably an oligonucleotide probe having the nucleotide sequence represented by SEQ ID NO: 30. An oligonucleotide probe (p-4) is an oligonucleotide probe having a nucleotide sequence of 10 to 30 consecutive nucleotides including nucleotides at positions 192 to 211 of SEQ ID NO: 12 in which G at position 102 is substituted with T and A at position 105 is substituted with G (preferably including the nucleotides represented by SEQ ID NO: 31). The probe is preferably an oligonucleotide probe having the nucleotide sequence represented by SEQ ID NO: 31.

### Oligonucleotide Probe Group (q):

An oligonucleotide probe (q-1) is an oligonucleotide probe having a nucleotide sequence of 10 to 30 consecutive nucleotides including nucleotides at positions 230 to 249 of SEQ ID NO: 6, 11, 12, 13, 14, or 15 (preferably including the nucleotides represented by SEQ ID NO: 34). The probe is preferably an oligonucleotide probe having the nucleotide sequence represented by SEQ ID NO: 34. An oligonucleotide probe (q-2) is an oligonucleotide probe having a nucleotide sequence of 10 to 30 consecutive nucleotides including nucleotides at positions 230 to 249 of SEQ ID NO: 4, 5, or 9 (preferably including the nucleotides represented by SEQ ID NO: 35). The probe is preferably an oligonucleotide probe having the nucleotide sequence represented by SEQ ID NO: 35.

### Oligonucleotide Probe Group (r):

An oligonucleotide probe (r-1) is an oligonucleotide probe having a nucleotide sequence of 10 to 30 consecutive nucleotides including nucleotides at positions 248 to 267 of SEQ ID NO: 2, 4, 5, 6, 9, 10, 11, 12, 13, or 14 (preferably including the nucleotides represented by SEQ ID NO: 36). The probe is preferably an oligonucleotide probe having the nucleotide sequence represented by SEQ ID NO: 36. An oligonucleotide probe (r-2) is an oligonucleotide probe having a nucleotide sequence of 10 to 30 consecutive nucleotides including nucleotides at positions 248 to 267 of SEQ ID NO: 15 (preferably including the nucleotides represented by SEQ ID NO: 37). The probe is preferably an oligonucleotide probe having the nucleotide sequence represented by SEQ ID NO: 37. An oligonucleotide probe (r-3) is an oligonucleotide probe having a nucleotide sequence of 10 to 30 consecutive nucleotides including nucleotides at positions 248 to 267 of SEQ ID NO: 2, 4, 5, 6, 9, 10, 11, 12, 13, or 14 in which C at position 256 is substituted with T and T at position 265 is substituted with A (preferably including the nucleotides represented by SEQ ID NO: 38). The probe is preferably an oligonucleotide probe having the nucleotide sequence represented by SEQ ID NO: 38. An oligonucleotide probe (r-4) is an oligonucleotide probe having a nucleotide sequence of 10 to 30 consecutive nucleotides including nucleotides at positions 248 to 267 of SEQ ID NO: 2, 4, 5, 6, 9, 10, 11, 12, 13, or 14 in which C at position 256 is substituted with T (preferably including the nucleotides represented by SEQ ID NO: 39). The probe is preferably an oligonucleotide probe having the nucleotide sequence represented by SEQ ID NO: 39.

### Oligonucleotide Probe Group (s):

An oligonucleotide probe (s-1) is an oligonucleotide probe having a nucleotide sequence of 10 to 30 consecutive nucleotides including nucleotides at positions 342 to 361 of SEQ ID NO: 3, 4, 5, 6, 7, 8, 9, 10, 11, 12, 13, 14, or 15 (preferably including the nucleotides represented by SEQ ID NO: 52). The probe is preferably an oligonucleotide probe having the nucleotide sequence represented by SEQ ID NO: 52. An oligonucleotide probe (s-2) is an oligonucleotide probe having a nucleotide sequence of 10 to 30 consecutive nucleotides including nucleotides at positions 342 to 361 of SEQ ID NO: 2 or 4 (preferably including the nucleotides represented by SEQ ID NO: 53). The probe is preferably an oligonucleotide probe having the nucleotide sequence represented by SEQ ID NO: 53.

In the above description, the expression “an oligonucleotide probe having a nucleotide sequence of 10 to 30 consecutive nucleotides including nucleotides at positions Y to Z of SEQ ID NO: X” indicates an oligonucleotide probe which is a partial sequence of 10 to 30 consecutive nucleotides of SEQ ID NO: X, and comprises nucleotides at positions Y to Z of SEQ ID NO: X.

Also, in the above description, the expression “an oligonucleotide probe having a nucleotide sequence of 10 to 30 consecutive nucleotides including nucleotides at positions Y to Z of SEQ ID NO: X in which v (base) at position n is substituted with w (base)” indicates an oligonucleotide probe which is a partial sequence comprising 10 to 30 consecutive nucleotides of SEQ ID NO: X in which v (base) at position n has been substituted with w (base) and comprises nucleotides at positions Y to Z of SEQ ID NO: X.

The lengths of the above oligonucleotide probes of the present invention are generally 10 to 30 nucleotides, preferably 15 to 25 nucleotides, and more preferably 17 to 23 nucleotides.

The oligonucleotide probes are preferably nucleic acids and more preferably DNAs. Such DNAs include double-strand and single-strand DNAs. However, the oligonucleotide probes of the present invention are preferably single-strand DNAs.

SEQ ID NO: 1 represents the nucleotide sequence of the IG region of the EU type. SEQ ID NO: 2 represents the nucleotide sequence of the IG region of the Af2 type. SEQ ID NO: 3 represents the nucleotide sequence of the IG region of the CY type. SEQ ID NO: 4 represents the nucleotide sequence of the IG region of the MY-a type. SEQ ID NO: 5 represents the nucleotide sequence of the IG region of the MY-c type. SEQ ID NO: 6 represents the nucleotide sequence of the IG region of the B1-c type. SEQ ID NO: 7 represents the nucleotide sequence of the IG region of the SC type. SEQ ID NO: 8 represents the nucleotide sequence of the IG region of the MY-e type. SEQ ID NO: 9 represents the nucleotide sequence of the IG region of the MY-d type. SEQ ID NO: 10 represents the nucleotide sequence of the IG region of the MY-b type. SEQ ID NO: 11 represents the nucleotide sequence of the IG region of the B2 type. SEQ ID NO: 12 represents the nucleotide sequence of the IG region of the B1-d type. SEQ ID NO: 13 represents the nucleotide sequence of the IG region of the B1-b type. SEQ ID NO: 14 represents the nucleotide sequence of the IG region of the B1-a type. SEQ ID NO: 15 represents the nucleotide sequence of the IG region of the Af3 type. SEQ ID NO: 16 represents the nucleotide sequence of the IG region of the Af1 type. In addition, the nucleotide sequence of the IG region of each genome type can be obtained from, for example, the gene database GenBank provided by the NCBI (National Center for Biotechnology Information).

The oligonucleotide probes of the present invention can be obtained by, for example, chemical synthesis with the use of a nucleic acid synthesizer. Examples of a nucleic acid synthesizer that can be used include apparatuses referred to as a DNA synthesizer, an automatic nucleic acid synthesizer, and a nucleic acid automatic synthesizer.

The set of oligonucleotide probes of the present invention is preferably used in the form of a microarray wherein probes are immobilized on a carrier. As materials used for a carrier, materials known in the art can be used without particular limitation. Examples thereof include: noble metals such as platinum, platinum black, gold, palladium, rhodium, silver, mercury, tungsten, and compounds thereof; conductor materials such as carbon represented by graphite and carbon fiber; silicon materials represented by single crystal silicon, amorphous silicon, silicon carbide, silicon oxide, silicon nitride, and the like; composite materials represented by SOI (silicon-on-insulator) and the like, comprising the above silicon materials; inorganic materials such as glass, silica glass, alumina, sapphire, ceramics, forsterite, and photosensitive glass; and organic materials such as polyethylene, ethylene, polypropylene, circularpolyolefin, polyisobutylene, polyethylene terephthalate, unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenol resin, urea resin, epoxy resin, melamine resin, a styrene-acrylonitrile copolymer, a acrylonitrile-butadiene styrene copolymer, polyphenylene oxide, and polysulfone. The form of a carrier is not particularly limited; however, it is preferably a plate form.

According to the present invention, a carrier having a carbon layer and a chemically modifying group on the surface thereof is preferably used. Such carrier having a carbon layer and a chemically modifying group on the surface thereof includes a carrier that has a substrate on the surface of which a carbon layer and chemically modifying groups are provided and a carrier that has a substrate made of a carbon layer and a chemically modifying group on the surface of the substrate. Materials known in the art can be used for such a substrate. Materials similar to the above listed for a carrier can be used, but are not particularly limited thereto.

For the microarray of the present invention, a carrier having a fine plate structure is preferably used. The shape of such carrier may be a rectangular, square, or a circular shape, but it is not limited thereto. In general, the carrier used has a 1- to 75-mm square shape, preferably a 1- to 10-mm square shape, and more preferably a 3- to 5-mm square shape. For the ease of production of a carrier having a fine plate structure, a substrate of a silicon material or a resin material is preferably used. Particularly preferably, a carrier comprising a substrate made of single crystal silicon, and a carbon layer and a chemically modifying group on the substrate is used. Single crystal silicon includes silicon in which some crystallographic axes are slightly disoriented (sometimes referred to as mosaic crystal) and silicon with an atomic-level disordered arrangement (lattice defect).

According to the present invention, preferred examples of a carbon layer formed on a substrate include, but are not particularly limited to, synthetic diamond, high-pressure synthetic diamond, natural diamond, soft diamond (e.g., diamond like carbon), amorphous carbon, carbon materials (e.g., graphite, fullerene, and carbon nanotube), mixtures thereof, and laminates thereof. In addition, carbides such as hafnium carbide, niobium carbide, silicon carbide, tantalum carbide, thorium carbide, titanium carbide, uranium carbide, tungsten carbide, zirconium carbide, molybdenum carbide, chromium carbide, and vanadium carbide may also be used. The term “soft diamond” used herein collectively means an imperfect diamond structure, which is a mixture of diamond and carbon, such as a so-called diamond like carbon (DLC). The mixture ratio thereof is not particularly limited. A carbon layer is advantageous in that it is superior in chemical stability and thus it can endure subsequent introduction of a chemically modifying group and a coupling reaction with an analyte, in that it binds to an analyte with flexibility as a result of electrostatic coupling, in that it does not absorb UV radiation and thus it is transparent to UV radiation used in a detecting system, and in that it can be energized upon electroblotting. Also, a carbon layer is advantageous in that nonspecific adsorption to it rarely occurs upon a coupling reaction with an analyte. As described above, a carrier having a substrate itself made of a carbon layer may be used.

According to the present invention, a carbon layer can be formed by a conventional method such as a microwave plasma CVD (chemical vapor deposit) method, an ECRCVD (electric cyclotron resonance chemical vapor deposit) method, an ICP (inductive coupled plasma) method, a DC sputtering method, an ECR (electric cyclotron resonance) sputtering method, an ionized evaporation method, an arc evaporation method, a laser evaporation method, an EB (electron beam) evaporation method, or a resistance heating evaporation method.

According to a high-frequency plasma CVD method, a raw material gas (methane) is degraded by inter-electrode glow discharge generated by high-frequency waves so that a carbon layer is synthesized on a substrate. According to an ionized evaporation method, thermal electrons generated by a tungsten filament are used for degradation and ionization of a raw material gas (benzene) and a carbon layer is formed on a substrate with the use of a bias voltage. Alternatively, according to an ionized evaporation method, a carbon layer may be formed with a mixed gas comprising hydrogen gas (1% to 99% by volume) and methane gas (99% to 1% by volume).

According to an arc evaporation method, a carbon layer can be formed in the following manner. A DC voltage is applied between a solid graphite material (a cathode evaporation source) and a vacuum chamber (an anode) such that arc discharge is induced in vacuo for generation of plasma of carbon atoms at a cathode. Then, a more negative bias voltage than that at an evaporation source is applied to a substrate such that carbon ions in plasma are accelerated toward such substrate.

According to a laser evaporation method, a carbon layer can be formed by irradiating a graphite target plate with, for example, Nd:YAG laser (pulse oscillation) light for fusion of such plate and allowing carbon atoms to accumulate on a glass substrate.

When a carbon layer is formed on a substrate surface, the thickness of a carbon layer is generally approximately from the thickness of the monolayer thereof to 100 μm. If the thickness is too thin, the surface of a base substrate might be partially exposed. Meanwhile, if the thickness is too thick, productivity deteriorates. Thus, the thickness is preferably 2 nm to 1 μm and more preferably 5 nm to 500 nm.

An oligonucleotide probe can be tightly immobilized on a carrier by introducing a chemically modifying group on the surface of a substrate on which a carbon layer is formed. The chemically modifying group to be introduced can be adequately selected by a person skilled in the art. Examples thereof include, but are not particularly limited to, an amino group, a carboxyl group, an epoxy group, a formyl group, a hydroxyl group, and an active ester group.

An amino group can be introduced by, for example, carrying out ultraviolet irradiation or plasma treatment on a carbon layer in an ammonia gas. Alternatively, an amino group can be introduced by carrying out ultraviolet irradiation on a carbon layer in a chlorine gas for chlorination, followed by ultraviolet irradiation in an ammonia gas. Also, an amino group can be introduced by inducing a reaction on a chlorinated carbon layer in a gas containing a polyamine such as methylenediamine or ethylenediamine.

A carboxyl group can be introduced by, for example, allowing an adequate compound to react with a carbon layer aminated as described above. Examples of a compound used for introduction of a carboxyl group include: a halocarboxylic acid represented by the following formula: X—R¹—COOH (where X represents a halogen atom and R¹ represents a divalent hydrocarbon group having 10 to 12 carbon atoms) such as chloracetic acid, fluoroacetic acid, bromoacetic acid, iodoacetic acid, 2-chloropropionic acid, 3-chloropropionic acid, 3-chloroacrylic acid, or 4-chlorobenzoic acid; a dicarboxylic acid represented by the following formula: HOOC—R²—COOH (where R² represents a single bond or a divalent hydrocarbon group having 1 to 12 carbon atoms) such as oxalic acid, malonic acid, succinic acid, maleic acid, fumaric acid, or phthalic acid; a polycarboxylic acid such as polyacrylic acid, polymethacrylic acid, trimellitic acid, or butanetetracarboxylic acid; a keto acid or aldehyde acid represented by the following formula: R³—CO—R⁴—COOH (where R³ represents a hydrogen atom or a divalent hydrocarbon group having 1 to 12 carbon atoms and R⁴ represents a divalent hydrocarbon group having 1 to 12 carbon atoms); a dicarboxylic acid monohalide represented by the following formula: X—OC—R⁵—COOH (where X represents a halogen atom and R⁵ represents a single bond or a divalent hydrocarbon group having 1 to 12 carbon atoms) such as succinic acid monochloride or malonic acid monochloride; and an acid anhydride such as phthalic anhydride, succinic anhydride, oxalic anhydride, maleic anhydride, or butane tetracarbonic anhydride.

An epoxy group can be introduced by, for example, allowing an adequate polyepoxy compound to react with a carbon layer aminated as described above. Alternatively, an epoxy group can be introduced by allowing organic peracid to react with a carbon=carbon double bond in a carbon layer. Examples of organic peracid include peracetic acid, perbenzoic acid, diperoxyphthalic acid, performic acid, and pertrifluoroacetic acid.

A formyl group can be introduced by, for example, allowing glutaraldehyde to react with a carbon layer aminated as described above.

A hydroxyl group can be introduced by, for example, allowing water to react with a carbon layer chlorinated as described above.

The term “active ester group” used herein means an ester group having a highly acid electron-withdrawing group on the alcohol side and activating a nucleophilic reaction; that is to say, an ester group having high reaction activity. Such ester group has an electron-withdrawing group on the alcohol side and is activated to a greater extent than alkylester. An active ester group has reactivity with an amino group, a thiol group, a hydroxyl group, and the like. More specifically, phenol esters, thiophenol esters, N-hydroxyamine esters, cyanomethyl ester, esters of heterocyclic hydroxy compounds, and the like are known as active ester groups having much higher activity than alkyl esters or the like. Furthermore specifically, examples of an active ester group include a p-nitrophenyl group, an N-hydroxysuccinimide group, a succinimide group, a phthalic imide group, and a 5-norbornene-2,3-dicarboxyimide group. Particularly preferably, an N-hydroxysuccinimide group is used.

An active ester group can be introduced by, for example, carrying out active esterification of a carboxyl group introduced as described above with a dehydration-condensation agent such as cyanamide or carbodiimide (e.g., 1-[3-(dimethylamino)propyl]-3-ethylcarbodiimide) and a compound such as N-hydroxysuccinimide. With such treatment, it is possible to form a group in which an active ester group such as an N-hydroxysuccinimide group is bound to the end of hydrocarbon group via an amide bond (JP Patent Publication (Kokai) No. 2001-139532 A).

The oligonucleotide probes of the present invention are dissolved in a spotting buffer such that a spotting solution is prepared. The spotting solution is dispensed into each well of a 96- or 384-well plastic plate and the dispensed portions are spotted on a carrier by means of a spotter or the like. Thus, a microarray on which the oligonucleotide probes are immobilized on a carrier can be produced. Alternatively, a spotting solution may be manually spotted by means of a micropipetter.

After spotting, incubation is preferably carried out in order to allow a binding reaction between oligonucleotide probes and a carrier to proceed. Incubation is carried out generally at −20° C. to 100° C. and preferably at 0° C. to 90° C. generally for 0.5 to 16 hours and preferably for 1 to 2 hours. It is desired that incubation be carried out in a highly humid atmosphere at a humidity of 50% to 90%, for example. Following incubation, washing is preferably carried out using a washing reagent (e.g., 50 mM TBS/0.05% Tween 20, 2×SSC/0.2% SDS solution, or ultrapure water) for removal of DNA unbound to a carrier.

The set of oligonucleotide probes of the present invention may be immobilized on a single carrier or on different carriers. However, oligonucleotide probes belonged to the same oligonucleotide probe group are preferably immobilized on a single carrier. Different types of oligonucleotide probes of the present invention may be immobilized on a carrier as long as the above conditions are satisfied.

The present invention also relates to a method for determining the genome type of JC virus infecting a test subject with the use of the above set of oligonucleotide probes or the microarray. The determination method of the present invention comprises the steps of: extracting DNA from a sample derived from a test subject; amplifying the nucleic acid encoding the IG region of the JC virus genome with the use of the extracted DNA as a template; and detecting the amplified nucleic acid with the use of the set of oligonucleotide probes or the microarray of the present invention.

A test subject is generally a human. A sample derived from a test subject is not particularly limited as long as such sample is expected to contain JC virus. Examples of a sample include: blood-associated samples (e.g., blood, serum, and plasma); humors such as lymph, perspiration, tears, saliva, urine, feces, ascites, and cerebrospinal fluid; and disrupted products and extracts of cells, tissue, and organs (e.g., heart, pancreas, liver, ovaries, lung, brain, bone marrow, lymph node, and kidneys). Preferably, urine and a blood-associated sample are used. A sample derived from a test subject is not necessarily collected directly from a human body. For instance, a sample collected from a urine stain or a blood stain may be used.

First, DNA is extracted from a sample collected from a test subject. A means for extraction is not particularly limited. For instance, a DNA extraction method using phenol/chloroform, ethanol, sodium hydroxide, CTAB, or the like can be used.

Next, an amplification reaction is carried out using the obtained DNA as a template so that the nucleic acid encoding the IG region of JC virus (preferably DNA) is amplified. Examples of an amplification reaction that can be applied include a polymerase chain reaction (PCR), LAMP (loop-mediated isothermal amplification), and ICAN (isothermal and chimeric primer-initiated amplification of nucleic acids). Upon amplification reaction, it is desired that labeling be carried out for identification of an amplified region. In such case, a method for labeling an amplified nucleic acid is not particularly limited. For instance, a method wherein a primer used for an amplification reaction is preliminarily labeled or a method wherein a labeled nucleotide is used as a substrate for an amplification reaction may be used. Examples of a labeling substance that can be used include, but are not particularly limited to, radioactive isotopes, fluorescent dyes, and organic compounds such as digoxigenin (DIG) and biotin.

In addition, this reaction system includes a buffer, a thermostable DNA polymerase, primer specific to the JC virus IG region, a labeled nucleotide triphosphate (specifically, fluorescent-labeled nucleotide triphosphate), nucleotide triphosphate, and magnesium chloride, which are necessary for nucleic acid amplification and labeling.

A primer used for an amplification reaction is not particularly limited as long as the IG region of JC virus is specifically amplified with such primer. A person skilled in the art can adequately design such primer. Examples thereof include a primer set containing:
Primer 1: 5′-CACAAGCTTTTTTGGACACTAACAGGAGG-3′ (SEQ ID NO: 71); and
Primer 2: 5′-GATTCTGCAGAAGACTCTGGACATGG-3′ (SEQ ID NO: 72).

A hybridization reaction of the above obtained amplified nucleic acid and the oligonucleotide probes of the present invention is carried out. Then, the amounts of nucleic acids hybridized to the individual oligonucleotide probes can be determined by label detection. When, for example, a fluorescent label is used, the signal intensity of a signal from such label can be quantified by detecting a fluorescent signal with a fluorescence scanner and analyzing the detected signal with the use of image analysis software. Further, the amplified nucleic acids hybridized to the individual oligonucleotide probes can be quantified by making a calibration curve with the use of a sample containing a known amount of DNA. Preferably, a hybridization reaction is carried out under stringent conditions. The term “stringent conditions” indicates conditions in which a specific hybrid is formed while a nonspecific hybrid is not formed. Such conditions involve, for example, a hybridization reaction at 50° C. for 16 hours and washing with 2×SSC/0.2% SDS at 25° C. for 10 minutes and with 2×SSC at 25° C. for 5 minutes.

For the above hybridization reaction, it is preferable to use a microarray on which the set of oligonucleotide probes of the present invention is immobilized on a carrier and to apply amplified nucleic acids to the microarray.

The method for determining genome types of JC virus of the present invention is carried out by comparing the amounts of the above amplified nucleic acids hybridized to the individual oligonucleotide probes in each oligonucleotide probe group. Specifically, ranking of the amounts (corresponding to label-derived signal intensities, for example) of amplified nucleic acids hybridized to the individual oligonucleotide probes is carried out in each oligonucleotide probe group. Ranking results for each oligonucleotide probe group can be specific or non-specific to a particular genome type. Thus, the genome type of JC virus infecting a test subject can be determined by judging whether or not a sample derived from the test subject has a ranking specific to a particular genome type.

Regarding currently known genome types of JC virus, the IG regions are amplified and the amplified nucleic acids are hybridized to the set of oligonucleotide probes of the present invention. Accordingly, the following characteristics relating to the amounts of amplified nucleic acids hybridized to the individual oligonucleotide probes are shown depending on probe groups.

TABLE 1

| Genome type of JC virus | Characteristic ranking |
|---|---|
| EU | f-2 > f-1, f-3 |
| Af1 | b-2 > b-1 |
| Af2 | f-3 > f-1, f-2 |
| SC | e-1 < e-2 or e-1 < e-3 |
| CY | a-2 > a-1 |
| MY | d-2 > d-1, d-3, d-4 |
| | d-1 ≈ d-3 (equivalent) |
| B1-a | h-2 > h-1, h-3, h-4 |
| | j-2 > j-3 > j-1 |
| B1-b | h-3 > h-1, h-2, h-4 |
| B1-c | k-3 > k-2 > k-1 |
| B1-d | i-2 > i-1 |
| B2 | c-2 > c-1 |
| | g-3 > g-1, g-2 |
| | l-2 > l-1 |

For instance, in the case of the oligonucleotide probe group (f), when the amount of an amplified nucleic acid hybridized to the oligonucleotide probe (f-2) is larger than any of the amounts of amplified nucleic acids hybridized to the oligonucleotide probes (f-1) or (f-3), it can be determined that JC virus infecting a test subject corresponds to the EU type.

Also, in the case of the oligonucleotide probe group (b), when the amount of an amplified nucleic acid hybridized to the oligonucleotide probe (b-2) is larger than that hybridized to the oligonucleotide probe (b-1), it can be determined that JC virus infecting a test subject corresponds to the Af1 type.

Further, in the case of the oligonucleotide probe group (h), it can be determined that JC virus infecting a test subject corresponds to the B1-a type when the amount of an amplified nucleic acid hybridized to the oligonucleotide probe (h-2) is larger than any of the amounts of amplified nucleic acids hybridized to the oligonucleotide probes (h-1), (h-3), or (h-4) while the following condition is satisfied in the oligonucleotide probe group (j): the amount of amplified nucleic acid hybridized to the oligonucleotide probe (j-2)>the amount of amplified nucleic acid hybridized to oligonucleotide probe (j-3)>the amount of amplified nucleic acid hybridized to the oligonucleotide probe (j-1).

JC viruses have been spread throughout human populations. Each genome type has a specific distribution area in the world. Thus, the place of origin of a test subject can be estimated by determining the genome type of JC virus infecting the test subject. Therefore, the present invention also relates to a method for estimating the place of origin of a test subject based on the JC virus genome type determined by the above method with the use of the set of oligonucleotide probes or the microarray of the present invention.

The distribution area of each genome type is described in detail in Proc Natl Acad Sci USA, 94: 9191-9196, 1997; J Gen Virol, 79: 2499-2505, 1998; and Review: “JC virus genotyping offers a new paradigm in the study of human populations, “Rev Med Virol; 14(3): 179-91 2004. The specific distribution of 12 representative genome types is as follows: EU type in Europe; Af1 type in West Africa; Af2 type in Africa and West Asia; Af3 type in Central Africa; SC type from South China to Southeast Asia; CY type in Northeast China, the Korean Peninsula, and Japan; MY type in Korea, Japan, and the USA; B1-a type in China and the Philippines; B1-b type in Central Asia, West Asia, and neighboring regions; B1-c type in South Europe; B1-d type from the Middle East to Greece and neighboring regions; and B2 type in India and neighboring regions (described in the table in J Mol Evol 54: 285-297, 2002).

In the East Asia region, distribution is as follows: EU type in Siberia; SC type from South China to Southeast Asia; CY type in West Japan, Northeast China, and Korea; MY type in Northeast Japan; B1-a type in the Philippines; and B1-b type in West China.

This description includes part or all of the contents as disclosed in the description of Japanese Patent Application No. 2006-329735, which is a priority document of the present application.

### DESCRIPTION OF THE PREFERRED EMBODIMENTS

### Example 1

### Preparation of a Carrier

A double DLC layers were formed on a 3-mm square silicon substrate by ionized evaporation under the following conditions.

TABLE 2

| | | First | Second | |
|---|---|---|---|---|
| | | layer | layer | |
| Raw material gas | CH₄ | 4.75 | 47.5 | (sscm) |
| | H₂ | 0.25 | 2.5 | (sscm) |
| Working pressure | | 3.0 | 8.0 | (Pa) |
| Substrate bias | DC voltage | 500 | 500 | (V) |
| | High-frequency output | 100 | — | (W) |
| Anode voltage | | 50 | 50 | (V) |
| Filament | Voltage | 7 | 7 | (V) |
| | Current | 22 | 22 | (A) |

An amino group was introduced into the obtained silicon substrate having a DLC layer on the surface thereof with the use of ammonia plasma under the following conditions.

TABLE 3

| | | | |
|---|---|---|---|
| Raw material gas | NH₃ | 30 | (sscm) |
| Working pressure | | 8.0 | (sscm) |
| Substrate bias | DC voltage | 500 | (Pa) |
| | High-frequency output | — | (W) |
| Anode voltage | | 50 | (V) |
| Filament | Voltage | 7 | (V) |
| | Current | 22 | (A) |

The silicon substrate was immersed in a 1-methyl-2-pyrrolidone solution containing 140 mM succinic anhydride and 0.1 M sodium borate for 30 minutes for introduction of a carboxyl group. Then, the silicon substrate was immersed in a solution containing 0.1 M potassium phosphate buffer, 0.1 M 1-[3-dimethylamino)propyl]-3-ethylcarbodiimide, and 20 mM N-hydroxy succinimide for 30 minutes for activation. Accordingly, a carrier comprising a silicon substrate, and a DLC layer and an N-hydroxy succinimide group serving as a chemically modifying group on the surface of the silicon substrate, was obtained.

### Example 2

### Preparation of a Microarray

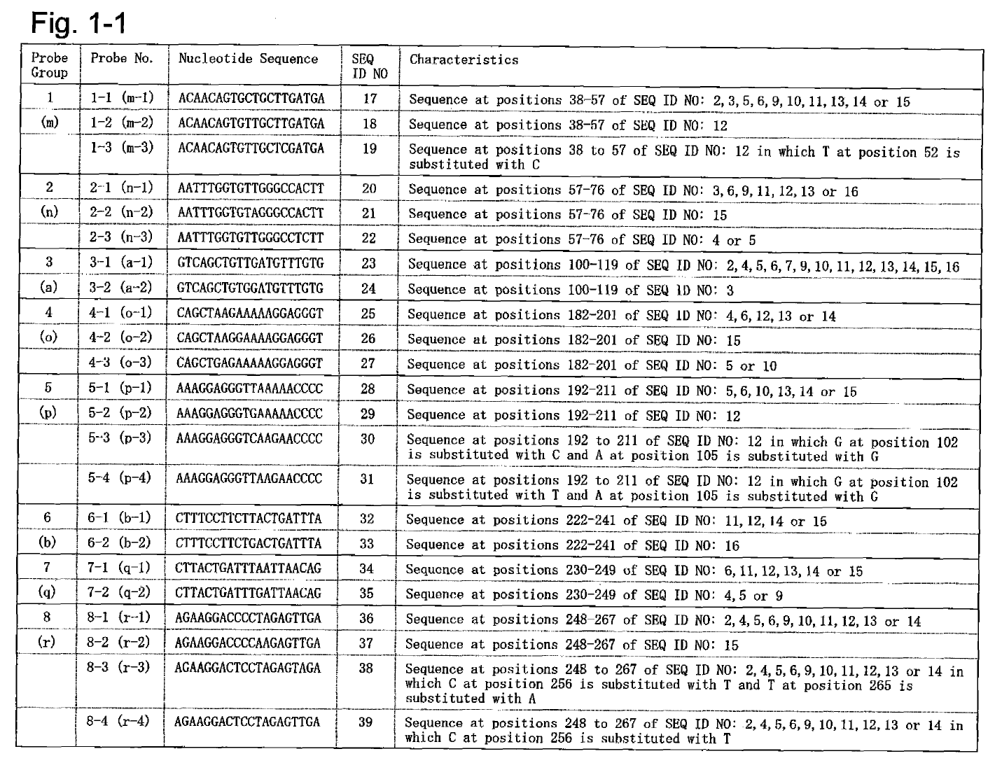

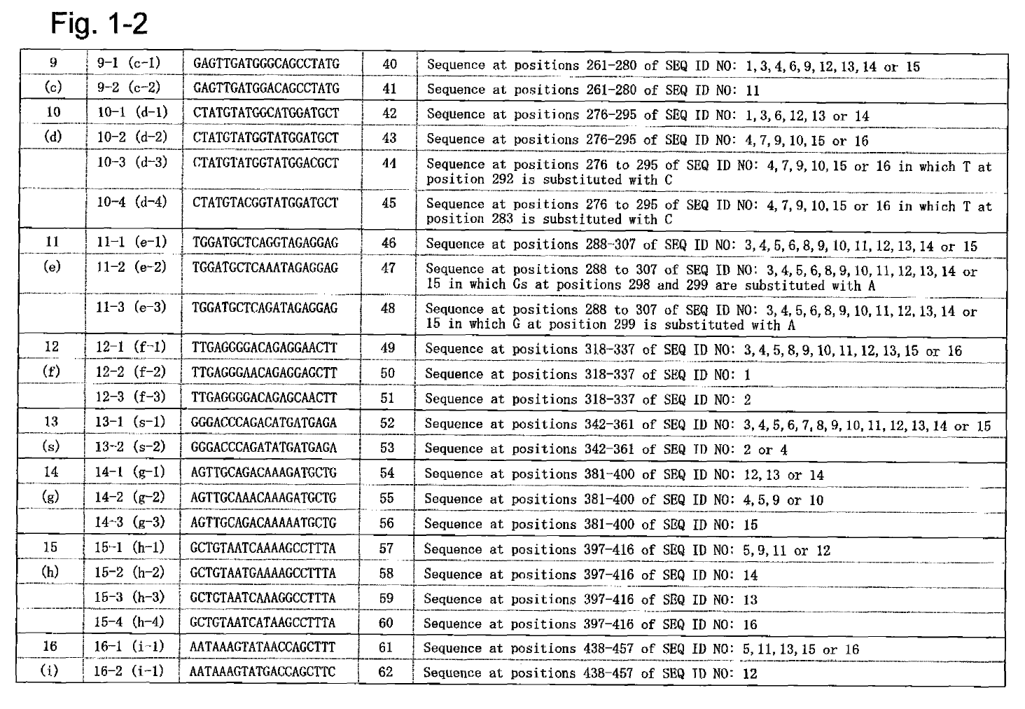

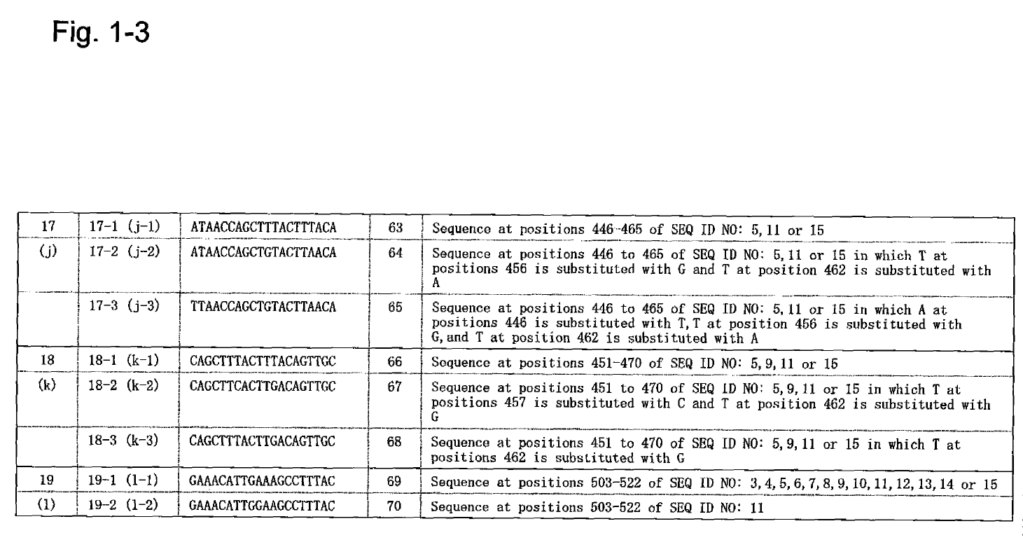

Based on the nucleotide sequences (SEQ ID NO: 1 to 16) of 16 different JC virus genome types, 54 types of oligonucleotide probes were synthesized, such probes each comprising 20 nucleotides and having the 5′ end modified with an amino group. FIG. 1 shows the sequences of the individual oligonucleotide probes (hereinafter referred to as probes). In FIG. 1, for each probe, the corresponding oligonucleotide probe group and the oligonucleotide probe of the present invention are described in brackets.

The 54 types of probes shown in FIG. 1 were dissolved in a spotting solution (Sol.6) at 10 pmol/μl and the obtained solution was spotted on the carrier prepared in Example 1 with the use of SPBIO (Hitachi Software Engineering Co., Ltd.). Baking was carried out at 80° C. for 1 hour, followed by washing with a 2×SSC/0.2% SDS solution (room temperature) for 15 minutes and further washing with a 2×SSC/0.2% SDS solution (95° C.) for 5 minutes. Then, rinsing was carried out 3 times with ultrapure water, followed by drying by a centrifuge for removal of water. Accordingly, a microarray supporting the carrier on which the 54 types of probes had been immobilized was obtained.

### Example 3

### Analysis of Signals Characteristic to JC Virus Genome Types

The microarray prepared in Example 2 was used for detection of a sample derived from a test subject with an identified JC virus genome type (EU, Af1, Af2, SC, CY, MY, B1-a, B1-b, B1-c, B1-d, or B2) (such sample being obtained by PCR amplification of the IG region with the use of DNA extracted from urine as a template and cloning of the amplified product in a plasmid vector).

DNA was extracted form the sample with the use of QiaAmp DNA (QIAGEN). A PCR reaction solution was prepared using the DNA as a template DNA in accordance with the following composition.

TABLE 4
Preparation of a PCR reaction solution (20.6 μL)

| | | |
|---|---|---|
| Primer 1 (10 μM) | 1 | μL |
| Primer 2 (10 μM) | 1 | μL |
| PCR buffer | 2 | μL |
| dNTP (dCTP: 1/10 concentration) | 2 | μL |
| Cy5-dCTP (Amersham Bio) | 0.5 | μL |
| Template DNA | 1 | μL |
| Ex Taq DNA polymerase (TAKARA Bio) | 0.1 | μL |
| H₂O | 13 | μL |

The sequences of the primers used were as follows.

(SEQ ID NO: 71)

| | |
|---|---|
| Primer 1: | 5′-CACAAGCTTTTTTGGACACTAACAGGAGG-3′ |

(SEQ ID NO: 72)

| | |
|---|---|
| Primer 2: | 5′-GATTCTGCAGAAGACTCTGGACATGG-3′ |

The prepared PCR reaction solution was subjected to a PCR reaction using a GeneAmp PCR system 9700 (ABI) at the following temperature cycle: 95° C. (0 seconds)→55° C. (0 seconds)→72° C. (10 seconds)→72° C. (1 minute)→4° C.

According to need, a 3×SSC/0.3% SDS solution (1 μL) was added to the sample subjected to a PCR reaction such that a target solution containing fluorescence-labeled target DNA was prepared.

The target solution was added dropwise to the microarray prepared in Example 2 and a hybridization cover was placed on the microarray. The microarray was allowed to stand at 50° C. for 30 minutes and then placed in a humidity bath for reaction. The cover was removed in a washing reagent, followed by rinsing with 2×SSC/0.2% SDS. Further rinsing with 2×SSC was carried out, followed by drying by centrifugation at 1500 rpm for 1 minute. Scanning was carried out using an FLA8000 fluorescence scanner (Fuji Photo Film Co., Ltd.). Based on a scanned image, signal intensities were quantified using the GenePixPro analysis software.

Signal analysis was carried out by comparing signal intensities and determining the ranking of signal intensities in each probe group. As a result, the following characteristic signals were detected depending on the individual genome types. It was revealed that it is possible to determine the JC virus genome type of an unidentified sample by determining the presence or absence of such characteristic signals.

TABLE 5

| JC virus genome type | Characteristic signal |
|---|---|
| EU | 12-2 > 12-1, 12-3 |
| Af1 | 6-2 > 6-1 |
| Af2 | 12-3 > 12-1, 12-2 |
| SC | 11-1 < 11-2 or 11-1 < 11-3 |
| CY | 3-2 > 3-1 |
| MY | 10-2 > 10-1, 10-3, 10-4 |
| | 10-1 ≈ 10-3 |
| B1-a | 15-2 > 15-1, 15-3, 15-4 |
| | 17-2 > 17-3 > 17-1 |
| B1-b | 15-3 > 15-1, 15-2, 15-4 |
| B1-c | 18-3 > 18-2 > 18-1 |
| B1-d | 16-2 > 16-1 |
| B2 | 9-2 > 9-1 |
| | 14-3 > 14-1, 14-2 |
| | 19-2 > 19-1 |

For instance, in the case of the genome type B1-a, when the result of comparison of signal intensities in a probe group 15 indicates 15-2>15-1, 15-3, and 15-4 (that is to say, when the largest signal intensity is 15-2) and the result of comparison of signal intensities in a probe group 17 indicates 17-2>17-3>17-1, an unidentified sample can be determined as corresponding to the genome type B1-a.

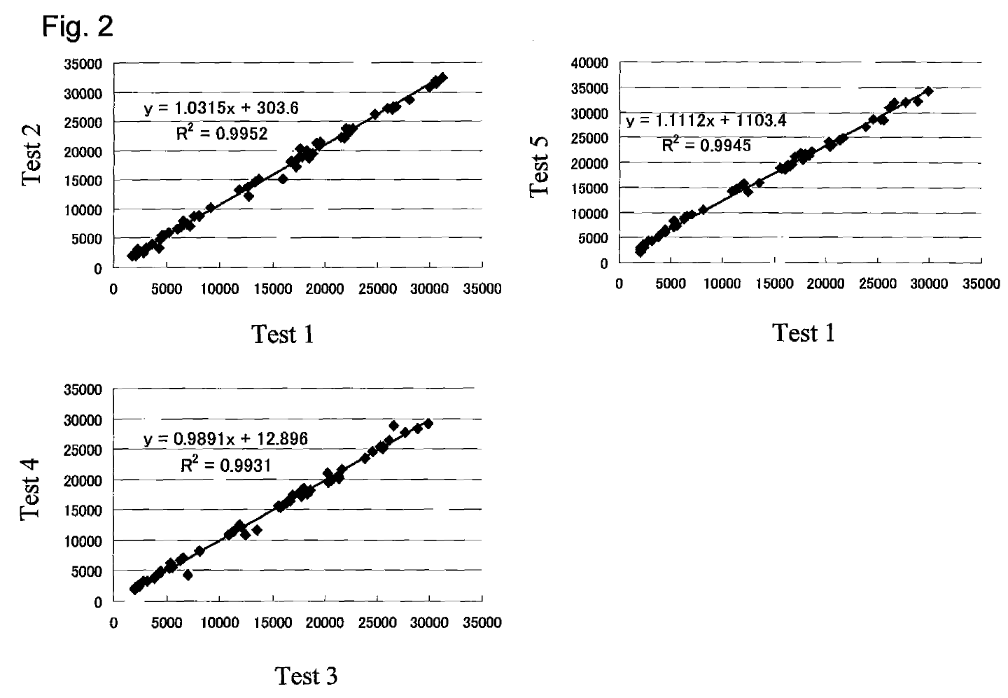

Further, FIG. 2 shows xy plotting results of fluorescence intensities obtained in two tests arbitrarily selected from among five tests with the use of identical samples. The figure shows that R² (correlation coefficient) was 0.99 or higher in each combination of two tests, indicating that the reproducibility of the test method is very high.

### Example 4

### Detection of the JC Virus Genome Type in an Unidentified Sample

DNAs were extracted using QiaAmp DNA (QIAGEN) from samples derived from test subjects with identified places of origins (kidney section: 20 mg; urine: 100 μL (urine stain: approximately φ 2 cm); or blood: 100 μL (blood stain: approximately φ 2 cm)). A PCR reaction solution was prepared using the DNA as a template DNA.

TABLE 6
Preparation of PCR reaction solution

| | | |
|---|---|---|
| Primer 1 (10 μM) | 0.5 | μL |
| Primer 2 (10 μM) | 0.5 | μL |
| Template DNA | 1.0 | μL |
| 10 × PCR buffer | 2.0 | μL |
| dNTP (dCTP: 1/10 concentration) | 2.0 | μL |
| Ex Taq DNA polymerase (Takara Bio) | 0.1 | μL |
| Cy5-dCTP (Amersham Bio) | 0.5 | μL |
| H₂O | 13.4 | μL |

The primers used were the same as those used in Example 3. The prepared PCR reaction solution was subjected to a PCR reaction using a GeneAmp PCR system 9700 (ABI) at the following temperature cycle so that the JC virus IG region of each sample was amplified:
95° C. (1 minute)→95° C. (10 seconds)→55° C. (10 seconds)→72° C. (30 seconds)→72° C. (2 minutes).

A 3×SSC/0.3% SDS solution (1 μL) was added to the sample (2 μL) subjected to a PCR reaction such that a target solution containing fluorescence-labeled target DNA was prepared.

The target solution was added dropwise to the microarray prepared in Example 2 and a hybridization cover was placed on the microarray. The microarray was allowed to stand at 50° C. for 30 minutes and then placed in a humidity bath for reaction. The cover was removed in a washing reagent, followed by washing 2 times with a 2×SSC/0.2% SDS solution and further washing 2 times with a 2×SSC solution and then drying by centrifugation for removal of water. Scanning was carried out using an FLA8000 fluorescence scanner (Fuji Photo Film Co., Ltd.). Based on a scanned image, signal intensities were quantified using the GenePixPro analysis software.

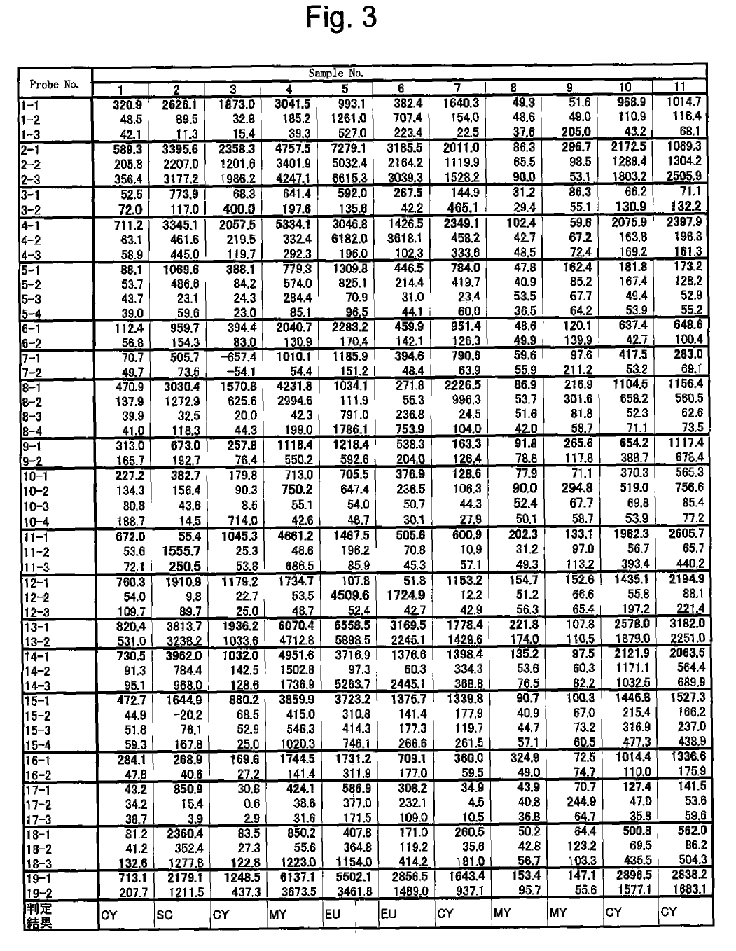

FIG. 3 shows signal intensity values obtained from each sample. In each probe group, a value corresponding to the strongest signal intensity obtained with the relevant probe is shown in bold. Also, a value corresponding to a signal characteristic to either one of the genome types is shown with hatching. The determination results are shown in the bottom row.

In the case of the results for a sample 1, the sample can be determined as corresponding to the CY type based on the signal intensities indicating “3-2>3-1” in the probe group 3. Also, signals in the other probe groups do not have characteristic of the other genome types. In addition, in the case of the results for a sample 2, the sample can be determined as corresponding to the SC type based on the signal intensities indicating “11-2>11-1, 11-3>11-1” in the probe group 11. Also, signals in the other probe groups do not have characteristic of the other genome types.

The same samples were subjected to a conventional method for determining JC virus genome types (a method for determining genome types by phylogenetic analysis, comprising determining the nucleotide sequence of an amplified IG region, NJ method, JOURNAL OF CLINICAL MICROBIOLOGY, June 1995, pp. 1448-1451) in a similar manner.

The results of genome typing are collectively shown in table 7.

TABLE 7

| | | | | Place of origin of test |
|---|---|---|---|---|
| | | Determination method | | subject (main JCV |
| Sample | | Micro- | Conventional | genome type in the |
| No. | Test sample | array | method | relevant area) |
| 1 | Blood | CY | CY | Chiba (CY ≧ MY) |
| 2 | Kidney | SC | SC | South Chiba (SC) |
| | section | | | |
| 3 | Kidney | CY | CY | Chiba (CY ≧ MY) |
| | section | | | |
| 4 | Kidney | MY | MY | Hokkaido (CY, MY) |
| | section | | | |
| 5 | Urine | EU | EU | Finland (EU) |
| 6 | Urine | EU | EU | Finland (EU) |
| 7 | Urine | CY | CY | Chiba (CY ≧ MY) |
| 8 | Urine | MY | MY | Akita (MY) |
| 9 | Urine stain | MY | MY | Iwate (MY) |
| | (1 week) | | | |
| 10 | Urine stain | CY | CY | Tottori (CY > MY) |
| | (1 week) | | | |
| 11 | Urine stain | CY | CY | Hyogo (CY > MY) |
| | (1 week) | | | |

Based on the above results, it has been shown that, according to the method of the present invention, it is possible to conveniently and accurately determine a genome type of JC virus infecting a test subject from a sample derived from the test subject so that the place of origin of the test subject can be posited.

All publications, patents, and patent applications cited herein are incorporated herein by reference in their entirety.

What is claimed is:

1. A set of oligonucleotide probes for determining the genome type of JC virus infecting a test subject, wherein the set containing the following oligonucleotide probes:
(a-1): an oligonucleotide probe having a nucleotide sequence of 10 to 30 consecutive nucleotides including nucleotides at positions 100 to 119 of SEQ ID NO: 2, 4, 5, 6, 7, 9, 10, 11, 12, 13, 14, 15, or 16;
(a-2): an oligonucleotide probe having a nucleotide sequence of 10 to 30 consecutive nucleotides including nucleotides at positions 100 to 119 of SEQ ID NO: 3;
(b-1): an oligonucleotide probe having a nucleotide sequence of 10 to 30 consecutive nucleotides including nucleotides at positions 222 to 241 of SEQ ID NO: 11, 12, 14, or 15;
(b-2): an oligonucleotide probe having a nucleotide sequence of 10 to 30 consecutive nucleotides including nucleotides at positions 222 to 241 of SEQ ID NO: 16;
(c-1): an oligonucleotide probe having a nucleotide sequence of 10 to 30 consecutive nucleotides including nucleotides at positions 261 to 280 of SEQ ID NO: 1, 3, 4, 6, 9, 12, 13, 14, or 15;
(c-2): an oligonucleotide probe having a nucleotide sequence of 10 to 30 consecutive nucleotides including nucleotides at positions 261 to 280 of SEQ ID NO: 11;
(d-1): an oligonucleotide probe having a nucleotide sequence of 10 to 30 consecutive nucleotides including nucleotides at positions 276 to 295 of SEQ ID NO: 1, 3, 6, 12, 13, or 14;
(d-2): an oligonucleotide probe having a nucleotide sequence of 10 to 30 consecutive nucleotides including nucleotides at positions 276 to 295 of SEQ ID NO: 4, 7, 9, 10, 15, or 16;
(d-3): an oligonucleotide probe having a nucleotide sequence of 10 to 30 consecutive nucleotides including nucleotides at positions 276 to 295 of SEQ ID NO: 4, 7, 9, 10, 15, or 16 in which T at position 292 is substituted with C;
(d-4): an oligonucleotide probe having a nucleotide sequence of 10 to 30 consecutive nucleotides including nucleotides at positions 276 to 295 of SEQ ID NO: 4, 7, 9, 10, 15, or 16 in which T at position 283 is substituted with C;
(e-1): an oligonucleotide probe having a nucleotide sequence of 10 to 30 consecutive nucleotides including nucleotides at positions 288 to 307 of SEQ ID NO: 3, 4, 5, 6, 8, 9, 10, 11, 12, 13, 14, or 15;
(e-2): an oligonucleotide probe having a nucleotide sequence of 10 to 30 consecutive nucleotides including nucleotides at positions 288 to 307 of SEQ ID NO: 3, 4, 5, 6, 8, 9, 10, 11, 12, 13, 14, or 15 in which Gs at positions 298 and 299 are substituted with A;
(e-3): an oligonucleotide probe having a nucleotide sequence of 10 to 30 consecutive nucleotides including nucleotides at positions 288 to 307 of SEQ ID NO: 3, 4, 5, 6, 8, 9, 10, 11, 12, 13, 14, or 15 in which G at position 299 is substituted with A;
(f-1): an oligonucleotide probe having a nucleotide sequence of 10 to 30 consecutive nucleotides including nucleotides at positions 318 to 337 of SEQ ID NO: 3, 4, 5, 8, 9, 10, 11, 12, 13, 15, or 16;
(f-2): an oligonucleotide probe having a nucleotide sequence of 10 to 30 consecutive nucleotides including nucleotides at positions 318 to 337 of SEQ ID NO: 1;
(f-3): an oligonucleotide probe having a nucleotide sequence of 10 to 30 consecutive nucleotides including nucleotides at positions 318 to 337 of SEQ ID NO: 2;
(g-1): an oligonucleotide probe having a nucleotide sequence of 10 to 30 consecutive nucleotides including nucleotides at positions 381 to 400 of SEQ ID NO: 12, 13, or 14;
(g-2): an oligonucleotide probe having a nucleotide sequence of 10 to 30 consecutive nucleotides including nucleotides at positions 381 to 400 of SEQ ID NO: 4, 5, 9, or 10;
(g-3): an oligonucleotide probe having a nucleotide sequence of 10 to 30 consecutive nucleotides including nucleotides at positions 381 to 400 of SEQ ID NO: 15;
(h-1): an oligonucleotide probe having a nucleotide sequence of 10 to 30 consecutive nucleotides including nucleotides at positions 397 to 416 of SEQ ID NO: 5, 9, 11, or 12;
(h-2): an oligonucleotide probe having a nucleotide sequence of 10 to 30 consecutive nucleotides including nucleotides at positions 397 to 416 of SEQ ID NO: 14;
(h-3): an oligonucleotide probe having a nucleotide sequence of 10 to 30 consecutive nucleotides including nucleotides at positions 397 to 416 of SEQ ID NO: 13;
(h-4): an oligonucleotide probe having a nucleotide sequence of 10 to 30 consecutive nucleotides including nucleotides at positions 397 to 416 of SEQ ID NO: 16;
(i-1): an oligonucleotide probe having a nucleotide sequence of 10 to 30 consecutive nucleotides including nucleotides at positions 438 to 457 of SEQ ID NO: 5, 11, 13, 15, or 16;
(i-2): an oligonucleotide probe having a nucleotide sequence of 10 to 30 consecutive nucleotides including nucleotides at positions 438 to 457 of SEQ ID NO: 12;
(j-1): an oligonucleotide probe having a nucleotide sequence of 10 to 30 consecutive nucleotides including nucleotides at positions 446 to 465 of SEQ ID NO: 5, 11, or 15;
(j-2): an oligonucleotide probe having a nucleotide sequence of 10 to 30 consecutive nucleotides including nucleotides at positions 446 to 465 of SEQ ID NO: 5, 11, or 15 in which T at position 456 is substituted with G and T at position 462 is substituted with A;
(j-3): an oligonucleotide probe having a nucleotide sequence of 10 to 30 consecutive nucleotides including nucleotides at positions 446 to 465 of SEQ ID NO: 5, 11, or 15 in which A at position 446 is substituted with T, T at position 456 is substituted with G, and T at position 462 is substituted with A;
(k-1): an oligonucleotide probe having a nucleotide sequence of 10 to 30 consecutive nucleotides including nucleotides at positions 451 to 470 of SEQ ID NO: 5, 9, 11, or 15;
(k-2): an oligonucleotide probe having a nucleotide sequence of 10 to 30 consecutive nucleotides including nucleotides at positions 451 to 470 of SEQ ID NO: 5, 9, 11, or 15 in which T at position 457 is substituted with C and T at position 462 is substituted with G;
(k-3): an oligonucleotide probe having a nucleotide sequence of 10 to 30 consecutive nucleotides including nucleotides at positions 451 to 470 of SEQ ID NO: 5, 9, 11, or 15 in which T at position 462 is substituted with G;
(l-1): an oligonucleotide probe having a nucleotide sequence of 10 to 30 consecutive nucleotides including nucleotides at positions 503 to 522 of SEQ ID NO: 3, 4, 5, 6, 7, 8, 9, 10, 11, 12, 13, 14, or 15; and
(l-2): an oligonucleotide probe having a nucleotide sequence of 10 to 30 consecutive nucleotides including nucleotides at positions 503 to 522 of SEQ ID NO: 11.

2. A microarray for determining the genome type of JC virus infecting a test subject, wherein the set of oligonucleotide probes according to claim 1 is immobilized on a carrier.

3. A microarray for estimating the place of origin of a test subject, wherein the set of oligonucleotide probes according to claim 1 is immobilized on a carrier.

4. The microarray according to claim 2, wherein the carrier has a carbon layer and a chemically modifying group on the surface thereof.

5. The microarray according to claim 3, wherein the carrier has a carbon layer and a chemically modifying group on the surface thereof.

6. A method for determining the genome type of JC virus infecting a test subject, comprising the steps of:
extracting DNA from a sample derived from a test subject;
amplifying the nucleic acid encoding the IG region of the JC virus genome with the use of the extracted DNA as a template; and
detecting the amplified nucleic acid with the use of the set of oligonucleotide probes according to claim 1.

7. A method for estimating the place of origin of a test subject based on the JC virus genome type determined by the method according to claim 6.

8. A method for determining the genome type of JC virus infecting a test subject, comprising the steps of:
extracting DNA from a sample derived from a test subject;
amplifying the nucleic acid encoding the IG region of the JC virus genome with the use of the extracted DNA as a template; and
detecting the amplified nucleic acid with the use of the microarray according to claim 2.

9. A method for estimating the place of origin of a test subject based on the JC virus genome type determined by the method according to claim 8.

10. A method for determining the genome type of JC virus infecting a test subject, comprising the steps of:
extracting DNA from a sample derived from a test subject;
amplifying the nucleic acid encoding the IG region of the JC virus genome with the use of the extracted DNA as a template; and
detecting the amplified nucleic acid with the use of the microarray according to claim 3.

11. A method for estimating the place of origin of a test subject based on the JC virus genome type determined by the method according to claim 10.

12. A method for determining the genome type of JC virus infecting a test subject, comprising the steps of:
extracting DNA from a sample derived from a test subject;
amplifying the nucleic acid encoding the IG region of the JC virus genome with the use of the extracted DNA as a template; and
detecting the amplified nucleic acid with the use of the microarray according to claim 4.

13. A method for estimating the place of origin of a test subject based on the JC virus genome type determined by the method according to claim 12.

14. A method for determining the genome type of JC virus infecting a test subject, comprising the steps of:
extracting DNA from a sample derived from a test subject;
amplifying the nucleic acid encoding the IG region of the JC virus genome with the use of the extracted DNA as a template; and
detecting the amplified nucleic acid with the use of the microarray according to claim 5.

15. A method for estimating the place of origin of a test subject based on the JC virus genome type determined by the method according to claim 14.
